(19) United States
(12) Patent Application Publication
(10) Pub. No.: US 2024/0148298 A1
(43) Pub. Date: May 9, 2024

(54) SYSTEMS AND METHODS FOR MONITORING ONE OR MORE ADDICTIVE ACTIVITIES OF AN ADDICT

(71) Applicant: LifeLine Medical LLC, Arlington, VA (US)

(72) Inventor: Christian Leon Haller, Arlington, VA (US)

(21) Appl. No.: 18/503,837

(22) Filed: Nov. 7, 2023

Related U.S. Application Data
(60) Provisional application No. 63/382,826, filed on Nov. 8, 2022.
(60) Provisional application No. 63/435,485, filed on Dec. 27, 2022.

(51) Int. Cl. A61B 5/16; A61B 5/00; A61B 5/11; G16H 80/00

(52) U.S. Cl. CPC A61B 5/165; A61B 5/0022; A61B 5/1112; A61B 5/4833; A61B 5/6831; A61B 5/6832; A61B 5/7264; A61B 5/7275; A61B 5/7282; A61B 5/746; A61B 5/7465; G16H 80/00; A61B 2560/0266

(57) ABSTRACT

Systems and methods for monitoring one or more addictive activities by an addict. A system includes at least at least one biosensor, a processor, and a communication system. The at least one biosensor measures physiological data from the addict. The processor generates a notification when the addict has participated in the one or more addictive activities based on the physiological data. The communication system sends the notification to one or more devices of the addict and/or of the members of an addiction support network of the addict to notify that the addict has participated in the addictive activities. The system uses geographical location or proximity data to notify the addict that the addict has entered a defined dangerous geographical zone or exited a defined safe geographical zone. The system includes a mobile system that automatically detects if the mobile system is being worn by the addict.

### CROSS-REFERENCE TO RELATED APPLICATIONS

This application claims priority to U.S. Provisional Application No. 63/382,826, filed Nov. 8, 2022, and to U.S. Provisional Application No. 63/435,485, filed Dec. 27, 2022, the entire contents of both of which are hereby incorporated by reference.

### TECHNICAL FIELD

The present disclosure relates generally to systems and methods for monitoring one or more addictive activities of an addict.

### BACKGROUND

An addict is a person that is addicted to certain substances or behaviors. Addicts participate in addictive activities including addictive substance abuse and/or participating in addictive behaviors. Often, an addict that participates in such addictive activities works with a sobriety partner to manage and end the addiction. A sobriety partner is a person who is continuously physically present with the addict, or is ready to be present with the addict, to intervene in the participation of addictive activities by the addict, to administer life-saving antidotes, to help the addict avoid potentially triggering locations or people, to ensure the addict stays within allowed locations at the appropriate times, and to engage the addict with ad hoc therapy.

### BRIEF SUMMARY OF THE INVENTION

In one aspect, a system for monitoring one or more addictive activities by an addict is disclosed. The system includes at least one biosensor that measures physiological data from the addict, a processor that generates a notification when the addict has participated in the one or more addictive activities based on the physiological data, and a communication system that sends the notification to one or more devices of the addict and/or of members of an addiction support network of the addict to notify that the addict has participated in the addictive activities.

In another aspect, a system for providing a virtual sobriety partner for an addict is disclosed. The system includes at least one biosensor that measures physiological data from the addict, and a processor that communicates with one or more devices of either a human or an artificial intelligence-based engine to notify the human or the artificial intelligence-based engine that the addict has participated in one or more addictive activities based on the physiological data.

In yet another aspect, a system for monitoring a risk of participation in one or more addictive activities by an addict using geographical location or proximity data is disclosed. The system includes at least one biosensor that measures physiological data from the addict, at least one geographical location sensor that measures geographical location or proximity data of the addict, a processor that generates a notification when the addict has either entered a defined dangerous geographical zone or exited a defined safe geographical zone, and a communication system that sends the notification to one or more devices of the addict to notify that the addict has entered the defined dangerous geographical zone or exited the defined safe geographical zone.

In yet another aspect, a mobile system for identifying an addict that uses the mobile system to prevent an unauthorized use of the mobile system by another other than the addict is disclosed. The mobile system includes at least one biosensor that measures physiological data from the addict, and a processor that sends, via a communication system, a notification to one or more devices of the addict and/or of members of an addiction support network of the addict that the mobile system is not being worn by the addict based on the physiological data

### BRIEF DESCRIPTION OF THE DRAWINGS

The foregoing and other features and advantages will be apparent form the following, more particular, description of various exemplary embodiments, as illustrated in the accompanying drawings, wherein like reference numbers generally indicate identical, functionally similar, and/or structurally similar elements.

FIG. 1 is a schematic diagram showing a breadth and scope of exemplary addictions, including both substances and behaviors, according to the present disclosure.

FIG. 2A is a schematic diagram of a living support bubble for an addict, according to the present disclosure.

FIG. 2B is a schematic diagram of a virtual support bubble for the addict of FIG. 2A, according to the present disclosure.

FIG. 3 illustrates a detailed, schematic diagram of a system for monitoring and detecting one or more addictive activities of an addict, according to the present disclosure.

FIG. 4 is a flow diagram showing an information process flow from a patient or wearer (e.g., an addict) to an addiction support network of the wearer, according to the present disclosure.

FIG. 5 is a flow diagram of a method for monitoring and detecting one or more addictive activities of an addict, according to the present disclosure.

FIG. 6 is a flow diagram of a method of monitoring and detecting one or more addictive activities of an addict, according to another embodiment.

FIG. 7 is a flow diagram of a method of monitoring and detecting one or more addictive activities of an addict, according to another embodiment.

FIG. 8A is a schematic illustration of a data processing workflow associated with signal preprocessing, data analysis, feature extraction, and predictive statistical modeling, according to the present disclosure.

FIG. 8B is detailed view of a predictive statistical analytics module of the data processing workflow of FIG. 8A, according to the present disclosure.

FIG. 9 is a schematic illustration of a data processing workflow 900, according to another embodiment.

FIG. 10 is a schematic illustration of two primary modes of communication between an addict and an addiction support network for the addict, according to the present disclosure.

FIG. 11 is a schematic illustration of a personal area network (PAN), according to the present disclosure.

FIG. 12 is a schematic illustration of one or more ring-based wearable devices, according to the present disclosure.

FIG. 13A is a schematic illustration of one or more adhesive-based wearable devices 1316, according to the present disclosure.

FIG. 13B is a schematic illustration of an adhesive-based wearable device, according to the present disclosure.

FIG. 14A is a schematic illustration of one or more band-mounted wearable devices, according to the present disclosure.

FIG. 14B is a detailed schematic illustration showing of a band-mounted wearable device, according to the present disclosure.

FIG. 15 is a detailed schematic illustration showing a band-mounted wearable device having a mobile-based software application, according to the present disclosure.

FIG. 16 is an illustrative table showing a generic state engine with various output conditions (as columns) and various biosensor-derived measurement parameters as input variables (as rows), according to the present disclosure.

FIG. 17 is a table showing a state engine for six exemplary conditions, showing addiction conditions as columns and various biosensor-derived measurements as input variables as rows, according to the present disclosure.

FIG. 18 is a table of biosensor-derived measurement parameters (as rows) collected over time after intervals of alcohol consumption (as columns, moving from left to right in time) for a single individual (N=1).

FIG. 19 is a table of biosensor-derived measurement parameters (as rows) collected over time after intervals of alcohol consumption (as columns, moving from left to right in time) for a second individual (N=1).

FIG. 20 is a table of biosensor-derived measurement parameters (as rows) collected over time after intervals of alcohol consumption (as columns, moving from left to right in time) for a third individual (N=1).

FIG. 21 illustrates an exemplary system that includes a general-purpose computing device, according to the present disclosure.

### DETAILED DESCRIPTION

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though they may. Furthermore, the phrases “in another embodiment” and “in some other embodiments” as used herein do not necessarily refer to a different embodiment, although they may. All embodiments of the disclosure are intended to be combinable without departing from the scope or spirit of the disclosure.

As used herein, “therapy” includes, without limitation, medical treatment of impairment, injury, disease, or disorder.

As used herein, “medical intervention” includes, without limitation, any drug, medical device, biological agent, or behavioral, mental, psychological, or psychiatric therapy given or engaged in by the subject of interest.

As used herein, “substance” includes, without limitation, any matter or material in any form.

As used herein, “behavior” includes, without limitation, an action, activity, or process that can be observed and measured.

As used herein “test” includes, without limitation, the assessment or diagnosis of a condition, physiological state or health condition, disease, or disorder.

As used herein, “intended use” refers to how any given test is going to get used, on what population, by which users, and in what context or environment.

As used herein, “biosensor” we mean an electromechanical or electrochemical device designed to measure a physical quantity, including but not limited to, photoplethysmography (PPG), Global Positioning System (GPS), electrodes, infrared temperature, imaging, molecular chemical detectors (MEMs, electrochemical), microphone, thermometer/thermistor, and/or terahertz spectroscopy.

As used herein, “real-time” is the actual time during which something takes place. The processors or servers detailed herein analyze data and perform the methods detailed herein as the data is received by the processors or servers, or can perform the methods detailed herein within a predetermined time interval, e.g., in the order of milliseconds or seconds. For example, “real-time” includes the processors or servers continuously or consistently receiving data from at least one biosensor and determining whether an addict is participating, or about to participate, in an addictive activity. Accordingly, the processors and servers detailed herein can perform the methods of the present disclosure as an addict is participating in addictive activity and/or is about to participate in an addictive activity.

As used herein, “addiction” includes, without limitation, compulsive dependence, the continued use of a mood-altering substance, or mood-altering behavior, despite adverse consequences.

As used herein, “physiologically altering substance” refers to a substance or activity which results in a physiological response.

As used herein, “mind-altering substances” are substances or activities which result in a psychoactive response, change in mood, state of mind, or level of pleasure.

As used herein, “physical addiction” refers to an increased tolerance for a substance or activity, that results in physical symptoms should one try to stop or reduce their intake substantially.

As used herein, “psychological addiction” refers to a mental dependence on substances or behaviors.

As used herein, a “wearer” is a person who wears the system and sensors of the present disclosure on their person. A “wearer” is also referred to herein as a “subject,” an “addict,” or an “addicted person.”

As used herein, an “addict” is a person that is addicted to one or more addictive activities (e.g., addictive substances and/or addictive behaviors). An “addict” is also referred to herein as a “wearer” or a “subject.”

As used herein, “addictive activity” and “addictive activities” include an addict consuming addictive substances or participating in addictive behaviors.

As used herein, “addictive substances” are external substances that lead to addiction after the external substance has entered the body of the addict and are used to achieve a desired psychological or physiologic addictive outcome. Non-limiting illustrative examples include, but are not limited to, legal drugs (e.g., opioids, benzodiazepines, or the like), illegal drugs (e.g., cocaine, fentanyl, amphetamines, or the like), foods (e.g., sweets, chocolate, pizza, etc.), beverages (e.g., alcoholic beverages, caffeinated beverages such as coffee, and sugary beverages such sodas, or the like), or any other substances to which a person can become addicted.

As used herein, “addictive behaviors” are those behaviors that lead to addiction without external substance entering the body, but rather release internal substances in the body to achieve a desired psychological or physiologic addictive outcome. Non-limiting illustrative examples include, but are not limited to, gambling, playing first-person shooter video games, watching pornography, engaging in excessive sex, running marathons, parachuting, or any other behaviors that achieve a desired psychological or physiologic addictive outcome.

As used herein, “onset of addiction” is a point in time in which termination of the addictive activity or consumption results in withdrawal or when tolerance for the consumed substance is developed.

As used herein, an “urge” is an intense desire to participate in an addictive activity, such as a desired to consume an addictive substance or engage in an addictive behavior. Moreover, urge is a form of stress, whereby cortisol levels shift, vasoconstriction occurs, blood pressure elevates, skin temperature increases, and/or galvanic skin conductance increases.

As used herein, “subjective self-report” is a subjective reporting on qualitative instruments of feelings, symptoms, emotions, and/or other opinions that one provides for themselves.

As used herein, “subjective third-party report” is a subjective reporting on qualitative instruments of feelings, symptoms, emotions, and/or other opinions that other people provide about a given subject of interest.

As used herein, a “user” is a person that consumes addictive substances or engages in addictive behaviors without the ability to control the use or consumption.

As used herein, “heavy user” is a person that consumes addictive substances or engages in addictive behaviors without the ability to control the use or consumption in a large or excessive quantity relative to the general population.

As used herein, “acute consumption” refers to sporadic or irregular consumption, with the limiting case of a single initial consumption, typically involving transient pharmacokinetic and pharmacodynamic changes that do not occur on a cyclical basis.

As used herein, “chronic consumption” refers to frequent or regular consumption or consumption on a regular or periodic basis. For example, chronic consumption can be as short as three (3) times depending on the dose and response of the subject to the consumed substance.

As used herein, “prescription compliance” refers to conformance to a dose and a frequency of a prescription.

As used herein, “withdrawal” refers to symptoms beginning to occur whenever the levels of a chemical fall in the bloodstream or the behavior is stopped.

As used herein, “pain” refers to significant discomfort sensed by the person from any physical, emotional, mental, or intellectual sources.

As used herein, “diagnosis” refers to any one of multiple intended uses of a diagnostic, including classifying subjects in categorical groups, aiding in the diagnosis when used with other additional information, screening at a high level where no a priori reason exists, when used as a prognostic marker, when used as a disease or injury progression marker, when used as a treatment response marker or as a treatment monitoring endpoint.

As used herein, “biomarker” is an objective measure of a biological or physiological function or process.

As used herein, “biomarker features” or “biomarker metrics” refer to a variable, a biomarker, a metric, or a feature which characterizes some aspect of the raw underlying time series data. Such terms are equivalent to a biomarker as an objective measure and can be used interchangeably.

As used herein, “non-invasively” refers to lacking the need to penetrate the skin or tissue of a subject.

As used herein, “adverse events” refer to any event negative to a person's health, well-being, or the continuation of life. For example, adverse events can include such minor events as the onset of a headache, muscle soreness, stomach upset, or can include much more significant events such as seizure, hospitalization, and ultimately death.

As used herein, “state engine” or “decision matrix” are equivalently any statistical predictive model with utilizes input factors X, typically measurements derived from biosensors on the body of a subject, to classify an unknown subject into a categorical or ordinal class or group Y, typically a substance or behavioral addictive condition, disease, or disorder.

As used herein, “susceptibility” refers to a genetic background that increases a person's probability to engage in substance or behavioral addiction.

As used herein, “occur” refers to events that may be in the future, are in the present, or have occurred in the past.

As used herein, “addiction support network” refers to individuals, collectively, that support an addict during a crisis and outside of crisis, including, but not limited to, first responders, EMTs, physicians, clinicians, recovery management specialists, psychologists, friends, family, chain-of-command, superiors, and/or direct reports.

As used herein, “artificial intelligence engine” refers to any type of analytical technique that includes signal processing along with statistical predictive modeling, including, but not limited to, machine learning, logistic regression, tree-based methods, discriminant analysis, fuzzy logic, or the like.

The present disclosure relates generally to systems and methods for monitoring and detecting detrimental activities of a subject. As used herein, monitoring and detecting can include monitoring for detrimental activities or can include both monitoring and detecting detrimental activities. For example, the present disclosure provides for systems and methods for detecting, predicting, and/or reporting aberrant and undesirable human behaviors and consumption of addictive substances, as well as to detect, monitor, or diagnose potential adverse physiological and psychological events resulting from these behaviors or substances.

A common problem today is the need to help people that suffer from addictions to various substances and behaviors. Addicts participate in addictive activities (e.g., addictive substance abuse and/or addictive behaviors) to pacify the addict's brain to feel better from some unresolved trauma, whether the unresolved trauma is mental or physical, real or imagined, or conscious or unconscious. There are very few efficient solutions, and none are cost-effective and may not accurately determine addictive activities or help to timely resolve the addictive activity. Accordingly, there is a need to have an inexpensive technology that could dramatically help addicts refrain from participating in addictive activities, such as, for example, consuming addictive substances or engaging in unwanted or addictive behaviors.

One method of managing and ending addiction is through the use of a sobriety partner, a person that is continuously physically present with the addict, to intervene in the consumption of addictive substances or participation in addictive behaviors, to administer life-saving antidotes in the case of adverse events, to help the addict avoid potentially triggering locations or people, to ensure the addict stays within allowed locations at the appropriate times, and/or to engage the addict with ad hoc therapy.

Currently, various detectors are used to measure a variety of physiological parameters, such as respiratory rate, respiratory depth, blood pressure, heart rate, blood oxygen, blood perfusion index, sinus rhythm, galvanic skin response, skin temperature, transdermal gases, other chemicals, and body and limb motion. One example detector is a type of detector designed to measure or sense pulse rate, blood oxygen level, and blood pressure via infrared sensors configured for photoplethysmography (PPG). There are numerous situations in which it may be desirable to use a detector to measure, analyze, and record human physiological activity and events. Further, there is a need to integrate one or more of these measurements and trends to assess and predict a selected human behavior or state of mind as it relates to the sum and interaction of the measurements. In practice, previous attempts to provide such a system to assess or predict human behavior have been unsatisfactory or inadequate, due to (1) the data not continuously streaming (e.g., real-time data transfer and analysis), (2) the systems not typically being wireless, (3) the systems not being portable, and/or (4) no real-time clinical assessment.

Additionally, such a system and measurements may be used to assess the clinical and emotional state of the wearer of the sensors. Further, such measurement may be used to provide detection of drug and alcohol consumption, medical adverse events, overdose, and participation in addictive activities and addictive behaviors. Such measurements may also be used to detect urges related to addictions such as drugs and alcohol, gambling, food, sex, and video games, or any other type of addiction.

Detection of urges, adverse events, consumption of addictive substances, participation in addictive activities, or predisposition to addiction, may be transmitted to caregivers, counselors, first responders, family, clinicians, or others as needed. Algorithms for analysis and detection may occur in a user wearable device, in a central location, in a cloud-based server, or a combination thereof.

Further understanding can be gained by considering the breadth of the problem to be resolved, for example, (1) opioid use becoming opioid addiction, (2) opioids with alcohol becomes an overdose condition, (3) prescription compliance is irregular, (4) recovery compliance is low and unpredictable, (5) continuous monitoring by a sobriety partner is generally the most effective tool for recovering addicts, (6) early detection of lapse through consumption of addictive substances or participation in addictive activities, can prevent a complete relapse of the addicted person, (7) detection of participation in addictive behaviors, allows for interdiction by appropriate parties.

Further, acute and chronic consumption, and withdrawal from addictive substances, or participation in addictive activities or behavioral disorders, present unique physiological data, and a different clinical approach may be indicated for each of these modes.

Accordingly, there is a need for new and improved systems and methods for integrating the required sensor data into a clinical diagnostic along with specific algorithms to provide real-time clinical feedback to all appropriate personnel and interested parties, such as, for example, one or more members of an addict's addiction support network.

The present disclosure comprises the provision and use of a new and improved system and apparatus for producing real-time, continuous data transmission and real-time clinical assessment of the human physiological response, behavior, and state of mind.

In one embodiment, the present disclosure consists of a system for monitoring for, and detecting, participation of addictive activities by a subject (e.g., an addict), including the consumption of addictive substances or participation in addictive behaviors by the subject. The system of the present disclosure comprises at least one biosensor that measures and records physiological data from the subject, a central processing unit that analyzes the physiological data in real-time and determines when the subject has participated in addictive activities (e.g., consumed addictive substances or participated in addictive behaviors), a communication system that notifies the subject and/or their addiction support network of the detrimental activity, and the system provides means for either uni-directional (e.g., 1-way) or bi-directional (e.g., 2-way) communication from the subject which intervenes and improves the care of the subject.

In other embodiments, the system has one or more biosensors attached to a wearer or subject. The biosensors can be combined into one or more housings and then either attached to or worn by a subject. In some embodiments, the housing is worn on the subject's wrist like a watch. In other embodiments, the biosensors can be adhesively or mechanically attached to the wearer. Other key elements include the ability of the biosensors to transmit sensor signals to a server via wireless communication or wired communication.

In some embodiments, the server processes biological sensor data received from the electronic module to identify and characterize artifacts, extract candidate features for classification and storage and/or for comparison to previously acquired candidate features, and generate a report. Naturally, the server algorithm is designed to determine if the subject has participated in addictive activities (e.g., participated in addictive behavior or consumed addictive substances). The system can further determine when the subject exhibits signs of addictive withdrawal, addictive cravings, or urges.

The system can further determine if the subject is compliant with a medical prescription from the wearer's care team. In one embodiment, the server could determine if the subject is exhibiting signs of detoxification or withdrawal of the addictive behavior or substance. The system can further determine if a subject is experiencing adverse events from the consumption of addictive substances or engaging in addictive behaviors. Moreover, the system can determine if a subject is engaged in a secondary support associated activity, such as withdrawing cash from an ATM proximal to a high-risk location. The server can determine if biosensor data should be sent to select members of the wearer's addiction support network, for instance, their clinicians and first responders. Moreover, the server can determine if alarms should be displayed on the wearer's device or transmitted to members of their addiction support network.

In some embodiments, the communication system can have single-way communication or bi-directional communication with the subject (e.g., with one or more devices on the subject or associated with the subject), or communication with an artificial intelligence-driven human-like avatar or bot. The system can conduct automated analysis to determine when a parameter is out of range due to the subject's participation in addictive activities (e.g., the consumption of an addictive substance or the participation in an addictive behavior) and then can automatically trigger the communication system to have uni-directional (e.g., 1-way) or bi-directional (e.g., 2-way) communication with the subject and their addiction support network.

In certain specialized addictive substances cases, the system can conduct an auto-injection of Narcan or other antidote substances to counter the consumption of addictive substances.

The system can monitor the physiological parameters with time series analysis including, but not limited to, logistic regression/classification, discriminant analysis, tree-based methods, fuzzy logic, genetic algorithms, machine learning, support vector machines, or any other predictive statistical method or model.

Further, the system can intervene if the subject has participated in addictive activities (e.g., consumed addictive substances or engaged in addictive behaviors) to disable heavy machinery, cars, planes, trains, boats, or dangerous equipment around the subject. After participation of addictive activities, the system can determine that a subject is not fit-for-duty and then notify the addiction support network of the subject's participation in the addictive activities.

In some embodiments, the system acts as a virtual sobriety partner. In such embodiments, there is at least one biosensor that measures and records physiological data from the subject, a central processing unit that analyzes the physiological data in real-time and determines when the subject has consumed addictive substances or participated in addictive behaviors, the means to notify the subject of the potential consumption or participation, the means to engage in 2-way communication with either a human or an artificial intelligence-based recovery engine serving as a virtual avatar for the subject, as well as the means to intervene with the subject to prevent or to minimize participation in the addictive activity (e.g., substance consumption or behavior participation), and optionally conduct advanced therapy with the subject. In some embodiments, the system can administer advanced therapy to the subject in the form of cognitive-behavioral therapy, talk therapy, self-management and recovery training (SMART) recovery or cost-benefit therapy, and 12-step therapy.

In some embodiments, the system can be configured for monitoring, and detecting, the risk of participation in addictive activities (e.g., consumption of addictive substances or participation in addictive behaviors) by a subject using geographical location or proximity data. In such embodiments, the system includes at least one biosensor that measures and records physiological data from the subject, at least one geographical location sensor that measures and records geographical location or proximity data from the subject, a central processing unit that analyzes the geographical location data in real-time and determines when the subject has either entered a defined dangerous geographical zone or exited a defined safe geographical zone, the means to notify the subject of the geographical breach, alternatively has the means to notify the subject's addiction support network of the geographical breach to enable intervention and the minimization of detrimental substance consumption or behavior participation.

In some embodiments, the system analysis combines geographical location or proximity data with physiological adverse event data to notify first responders to intervene and improve health. The geographical location sensor could be a Global Positioning System sensor (GPS), a Bluetooth transceiver, a Wi-Fi transceiver, and/or an ultra-wideband location sensor or equivalent. Another aspect enables the system to define both red zones (e.g., dangerous) or green zones (e.g., safe geographical zones) in both space and time, based on the individual subject's needs, and can change depending on the time of day as well as spatial proximity for both fixed and moving risks, such as a spouse or a drug dealer or a highway rest area. The analysis can determine if the subject is in a place conducive to consuming addictive substances or engaging in or supporting addictive behaviors. The system can transmit geographical location information to the subject's addiction support network which enables intervention when the subject is in crisis.

In another embodiment, the present disclosure consists of a mobile system that can uniquely identify the subject of the system to prevent the unauthorized use of the system by another person other than the subject. Such a system is achieved using at least one biosensor that measures and records physiological data from the subject, a central processing unit that analyzes the physiological data in real-time and determines when the system is not being worn by the subject, the means to notify the subject, and/or their addiction support network that the system is no longer being worn by the subject, provides means for either 1-way or 2-way communication from the subject which enables intervention and restoration of the system on the subject. In some embodiments, the system conducts a comparison to baseline measurements on the subject that can determine if the system is not being worn by the intended subject or by anyone.

The present disclosure also includes methods for measuring biological data using such devices. These and other characteristic features of the present disclosure will become apparent to those skilled in the art from the following description of the exemplary embodiments.

A multi-modal physiological assessment system and methods enable the simultaneous recording and then subsequent analysis of multiple data streams of biological signal measurements to monitor and/or to detect the consumption of addictive substances or participation in addictive behaviors by a subject. It comprises at least one biosensor that measures and records physiological data from the subject, a central processing unit that analyzes the physiological data in real-time, a communication system with the means to notify the subject and/or their addiction support network 1-way or 2-way communication from the subject which intervenes and improves the care of the subject. It enables the use of geographical location or proximity data to preemptively monitor the subject's spatial and temporal movements to attempt to keep them in green safe zones and out of red danger zones. The system can automatically detect if it is being worn by the proper subject if worn by a human at all.

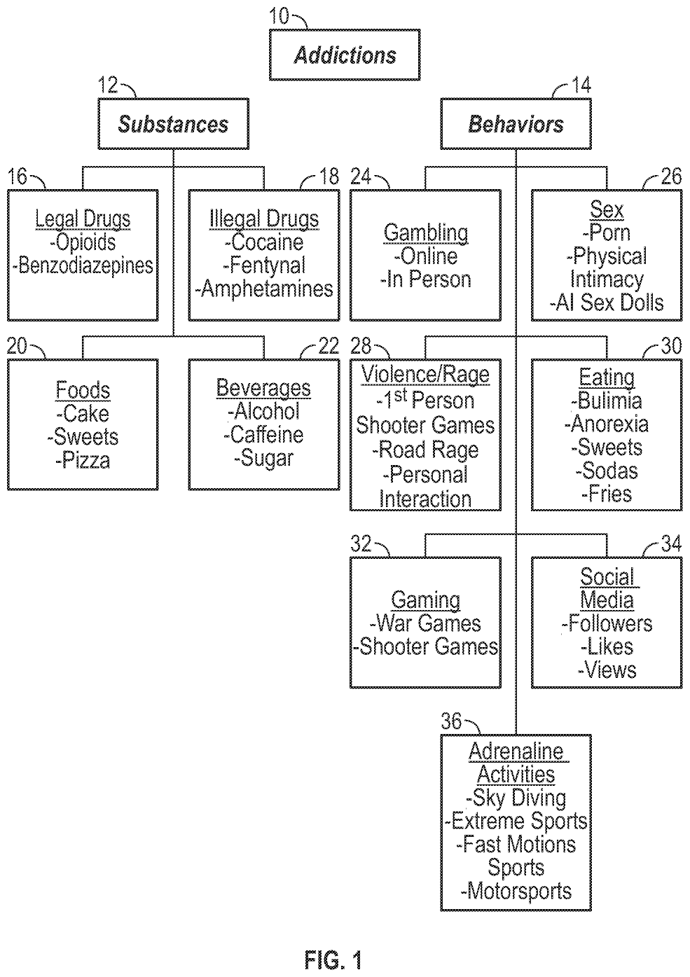

Referring now to the drawings, FIG. 1 is a schematic diagram showing a breadth and scope of exemplary addictions 10, including both substances and behaviors, according to the present disclosure. The systems and methods of the invention comprise devices and equipment form factors that can easily be positioned on the human body to both collect a multitude of biosignals as well as monitor and coordinate care for a subject. The field of addictions 10 can be organized into two major categories as shown in FIG. 1, including addictions related to substances 12 (e.g., addictive substances) as well as those related to certain behaviors 14 (e.g., addictive behaviors). The substances 12 related to addiction can further be subdivided into a sub-category of legal drugs 16, a sub-category of illegal drugs 18, a sub-category of foods 20, and a sub-category of beverages 22. The sub-category of legal drugs 16 includes, but is not limited to, opioids, central nervous system (CNS) depressants, stimulants, sildenafil, alcohol, tobacco, nicotine, caffeine, benzodiazepines, tetrahydrocannabinol (THC), or the like. The sub-category of illegal drugs 18 includes, but is not limited to, opioids such as heroin, cocaine, fentanyl, amphetamines, THC, ecstasy, pain relievers, depressants, stimulants, cocaine, inhalants, or the like. The sub-category of foods 20 includes, but is not limited to, sugar or sugary food products including cakes, pies, brownies, cookies, doughnuts, Twinkies™, Ho Hos™, Ding Dong's™, Pop Tarts™, and other corn syrup, sucrose, fructose, or other sweetener-based food products, salt and salty foods, fat and fatty foods, or the like. The sub-category of beverages 22 includes, but is not limited to, addictive substances such as alcohol, caffeine, sugars as listed above, including, but not limited to, natural and artificial sweeteners, corn syrup, honey, maple syrup, and other tree-based products, or the like.

In addition to the substances 12, addictions 10 consist of a set of behaviors 14 (e.g., addictive behaviors) that can be further divided into various sub-categories, such as, for example, a sub-category of gambling 24, a sub-category of sex 26, a sub-category of violence or rage 28, a sub-category of eating 30, a sub-category of gaming 32, a sub-category of social media 34, and a sub-category of adrenaline activities 36. The sub-category of gambling 24 includes both in-person gambling, for example, in Las Vegas, Atlantic City, Monte Carlo, etc., and also online or mobile app-based gambling related to professional and college sports, horse racing, national, state, and local elections, or anything else of an uncertain nature including the weather, or the like. The sub-category of sex 26 includes, but is not limited to, the consumption of pornography, engaging in casual sex, engaging in prostitution, and even including such exotic objects as sex dolls. The sub-category of sex 26 also includes dial-up telephonic and video services in which explicit discussion of sexually related material would take place with either a human being or a technology avatar. The sub-category of violence and rage 28 includes, but is not limited to, first-person shooter video games, such as Minecraft, League of Legends, Grand Theft Auto, War of Warcraft, Halo, and Grim Reaper, as well as engaging in paintball and other violence oriented physical activities. The sub-category of violence or rage 28 also includes addictive behaviors that are associated with rage, including, but not limited to, road rage as well as the rage from intense one-on-one personal interaction with another person. The sub-category of eating 30 includes, but is not limited to, anorexia, bulimia, consumption of sweets, sodas, and french fries, or the like. The sub-category of gaming 32 includes, but is not limited to, war games, shooter games, as well as multi-level games like Pac-man, Minecraft, League of Legends, Grand Theft Auto, War of Warcraft, Halo, Grim Reaper, Suduko, or the like. The sub-category of social media 34 in which people post content and look for social acceptance and feedback in the forms of likes, followers, and views. Those who post on social media can get wrapped up in getting more and more positive feedback until it becomes an addiction and reliance on social acceptance. The sub-category of adrenaline activities 36 includes addictive behaviors of people that are in pursuit of an adrenaline rush, so-called adrenaline junkies who engage in many different extreme sports looking for a periodic release of adrenaline. For example, the sub-category of adrenaline activities 36 includes, but is not limited to, skydiving, extreme skiing, hang gliding, wingsuit flying, cliff jumping, motorsports with high power to weight ratios, water motorsports on jet skis, wave runners, and other high power to rate ratio watercraft and aircraft, or the like. The systems and methods of the present disclosure can be further understood by the preferred embodiments shown in the drawings.

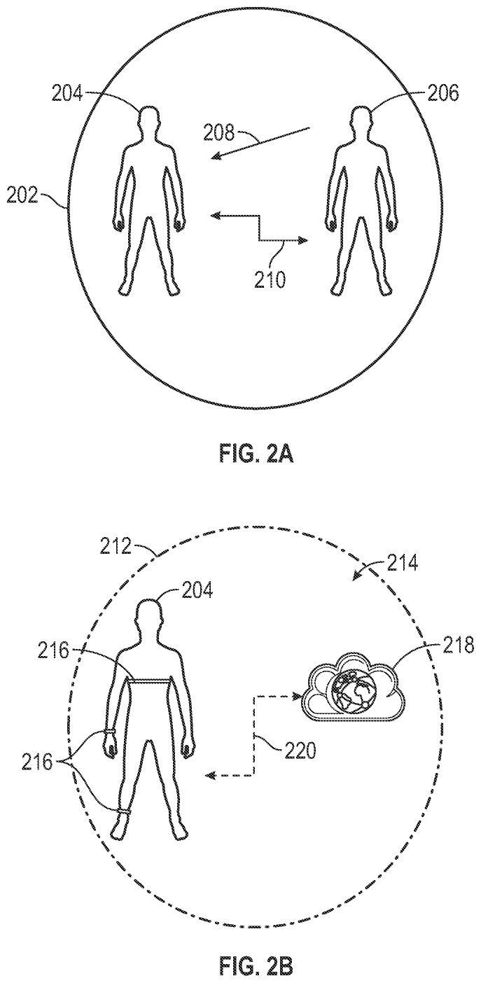

FIG. 2A is a schematic diagram of a living support bubble 202 for an addict 204, according to the present disclosure. When an alcoholic person is paired with a sobriety partner, the alcoholic person has a much better chance of recovery. While an alcoholic person is used herein as an example of an addict 204, the present disclosure can apply to any type of addict. FIG. 2A shows the addict 204 in recovery and living with a human sobriety partner 206 inside the living support bubble 202 whereby the two live as one living unit during a treatment and recovery process. The living support bubble 202 is an imaginary bubble that represents a support network that includes at least the addict 204 and the human sobriety partner 206, or other people that support the addict 204 in the treatment and recovery process. In the living support bubble 202, the human sobriety partner 206 lives in the same apartment or house as the addict 204 where the human sobriety partner 206 can continuously monitor (as indicated by the arrow 208) not only the consumption and behaviors of the addict 204, but also the physical location of the addict 204 to help keep the addict 204 away from bars, liquor stores, sports venues, or other locations that sell alcohol to adults under a state liquor license. Moreover, the human sobriety partner 206 can communicate (as indicated by arrow 210), through careful observation and training, to help the addict 204 identify for themselves “triggers” that send the addict 204 in search of alcohol. Additionally, the human sobriety partner 206 can provide 24/7 therapy through communication (as indicated by the arrow 210) with the addict 204 to help the addict 204 deal with their urges and addictive ingestion of alcohol. Such a model motivates the present disclosure and is schematically illustrated in FIG. 2B.

FIG. 2B is a schematic diagram of a virtual support bubble 212 for the addict 204, according to the present disclosure. In the embodiment of FIG. 2B, the human sobriety partner 206 of FIG. 2A is replaced with a virtual sobriety partner 214 that includes one or more wearable devices 216, also referred to as medical devices or mobile systems, that are worn by the addict 204 and are used as a “virtual sobriety partner” for the addict 204. The wearable device(s) are worn continuously (e.g., 24 hours a day, 7 days each week) and include one or more sensors therein for measuring and generating various information of the addict 204. Any removal or tampering with the wearable device(s) is internally detected and appropriately handled. Such a configuration creates the virtual support bubble 212 between the addict 204 and the virtual sobriety partner 214. In some embodiments, the virtual sobriety partner 214 includes a cloud-based network 218, also referred to as a cloud-based information technology infrastructure, that is also included in the virtual support bubble 212. The wearable devices 216 communicate with each other, as well as with the cloud-based network 218 through a data pipe 220. In this way, the virtual support bubble 212 provides for a system for monitoring and detecting one or more addictive activities of the addict 204.

Much like in the human-based model (FIG. 2A), in the medical device-based model (FIG. 2B), the sensors (e.g., in the wearable devices 216) in contact with the addict 204 can sense or observe both consumption and urges to participate in addictive activities (e.g., engage in ingestion of addictive substances and/or engage in addictive behaviors). Further, the medical device-based, virtual sobriety partner 214 can communicate with the addict 204 through a local display on one of the wearable devices 216 worn on the body of the wearer (e.g., the addict 204). In one embodiment, the one or more wearable devices 216 includes a smartwatch-like device that enables effective communication between the addict 204 and the virtual sobriety partner 214 through the cloud-based network 218. In some embodiments, actual humans within the virtual support bubble 212 (e.g., the addiction support network) of the wearer can send text messages and email messages, talk with the wearer as needed, or even conduct a video-based call. In some embodiments, the cloud-based network 218 includes automated bot-like pre-recorded messages that operate based on predetermined rules of engagement. The cloud-based network 218 can send the automated bot-like pre-recorded messages in an automated and scalable fashion to the wearable devices 216. The addiction support network consists of a broad host of people, including, but not limited to, crisis management-related first responders, clinicians, recovery management personnel, family, and friends that typically follow up with the addictive subject (e.g., the addict 204) when the addictive subject is either in or out of a crisis. The medical device-based approach of FIG. 2B can be used for any type of addiction, including all those associated with the consumption of substances as well as participation in addictive behaviors.

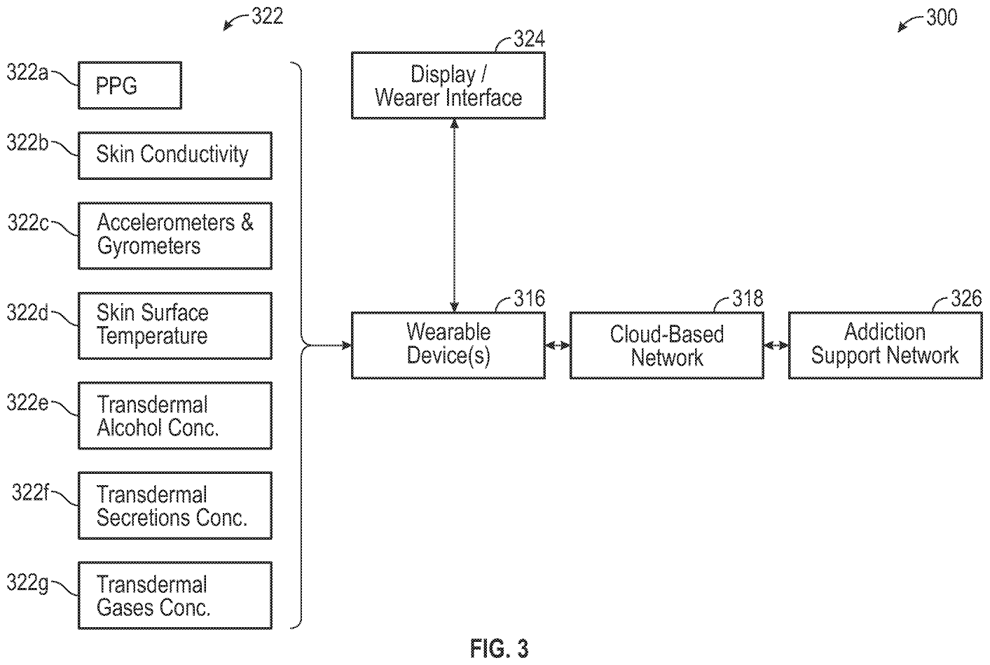

FIG. 3 illustrates a detailed, schematic diagram of a system 300 for monitoring and detecting one or more addictive activities of an addict, according to the present disclosure. The system 300 for monitoring and detecting can include monitoring for one or more addictive activities of the addict and/or can include both monitoring and detecting the one or more addictive activities of the addict. The system 300 can be utilized as the virtual sobriety partner 214 of FIG. 2B. The system 300 includes one or more wearable devices 316 having one or more biosensors 322. The one or more biosensors 322 includes, but is not limited to, a PPG sensor 322a, a skin conductivity sensor 322b (e.g., a transdermal conductivity sensor, an electrodermal activity sensor, or a galvanic skin conductance sensor), a many axis accelerometer and gyrometer sensor 322c, a skin surface temperature sensor 322d, a transdermal alcohol concentration sensor 322e, a transdermal secretion concentration sensor 322f, and a transdermal gas concentration sensor 322g.

The one or more biosensors 322 are contained in the one or more wearable devices 316. The one or more wearable devices 316 can clinically capture information from data streams of the one or more biosensors 322 and can display the information derived from the one or more biosensors 322 on a wearable interface 324. At the same time, the information from the one or more biosensors 322 is bidirectionally transferred to a cloud-based network 318, also referred to as a cloud-based information technology infrastructure, where members of an addict's addiction support network 326 can engage with the system 300 of the present invention. The addiction support network 326 for an addict includes several different individuals around the wearer (e.g., the addict). The list of individuals that comprise the addiction support network 326 of a wearer that can engage with the cloud-based network 318 includes, but is not limited to, people associated with crisis management, such as, for example, first responders, hotline operators, and emergency department clinicians, as well as individuals that typically follow up with the addicts when the addicts are not in crisis including family members, friends, recovery management personnel, other clinicians, including therapists that actively work to help the addict break their addiction.

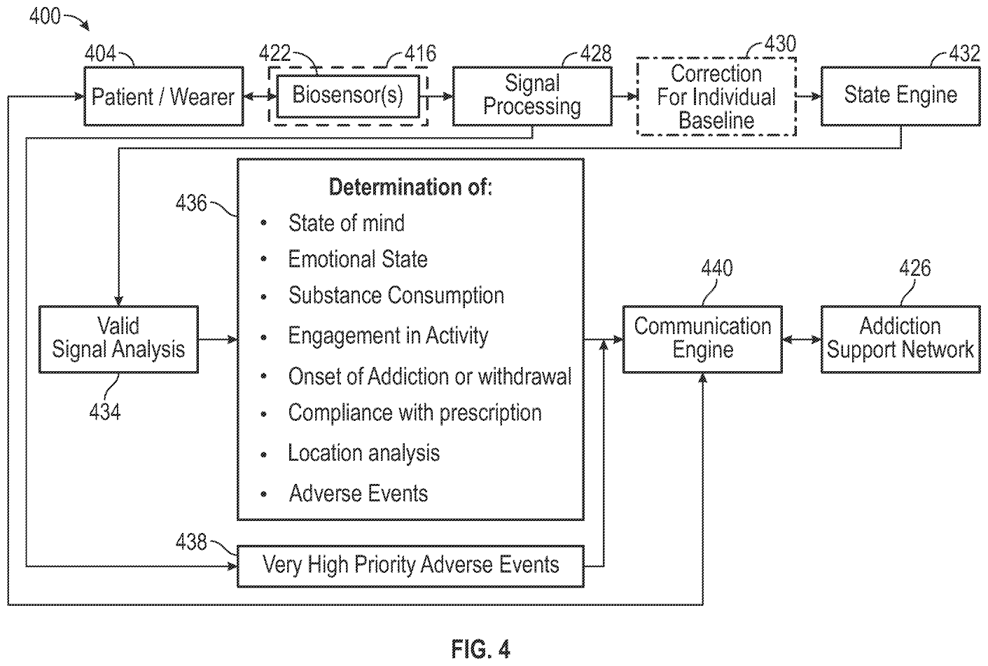

FIG. 4 is a flow diagram 400 showing an information process flow from a patient or wearer (e.g., an addict 404) to an addiction support network 426 of the addict 404, according to the present disclosure. When an addict requires a more precise diagnosis and ongoing monitoring according to the systems and methods of the present invention, the information flow can proceed as follows. As shown in FIG. 4, the addict 404 can wear one or more wearable devices 416 with one or more biosensors 422 that generate real-time biosensor data streams from the addict 404. The information flow can include signal processing 428 the real-time biosensor data streams. Optionally, an output from the signal processing 428 can undergo a correction for individual biological variability or baseline 430 before being entered into a state engine 432 of extracted features and other co-variates that take the various input factors, X, and either i) classify a subject into a group Y, or ii) conduct a regression to a numerical output score, Y. Either approach enables the patient, their family, their friends, their associated care team, and first responders to take care of the patient more comprehensively. In particular, the care team could initiate cognitive-behavioral therapy (CBT), discuss urges associated with triggers, discuss the consequences of relapse, discuss mindfulness, and/or discuss living in the present, with the patient.

An output from the state engine 432 is then tested for valid signal analysis 434 to ensure that artifact and error are not affecting the state engine 432 and associated predictive analytics. The present disclosure provides for the ability to make determinations 436 of a state of mind of the wearer, an emotional state of the wearer, a determination of any addictive substance consumed, or behavior engaged in by the wearer. The determinations 436 can further indicate the onset of addiction or even determine that the withdrawal process from an addictive substance or behavior is about to occur, occurring, or has occurred. The determinations 436 can conclude compliance, or lack thereof, with clinical prescriptions written by healthcare professionals to ameliorate the subject's addictive disorder. The determinations 436 can also include location analysis and determination of adverse events. The signal processing 428 can also flag possible very high probability adverse events 438 that may occur that could be expeditiously transferred to a communication engine 440 for automated communication to the addiction patient's addiction support network 426. The statistical predictions and the determinations 436 made from the output of the valid signal analysis 434 from the state engine 432 can then be combined with any flags related to the very high priority adverse events 438 and input into the communication engine 440 where information is relayed to the wearer's addiction support network 426 including members of the addiction support network 426 involved with both (i) crisis management (e.g., EMTs, doctors, first responders, or the like), as well as (ii) non-crisis participants, such as psychiatrists, family members, and friends of the patient or wearer.

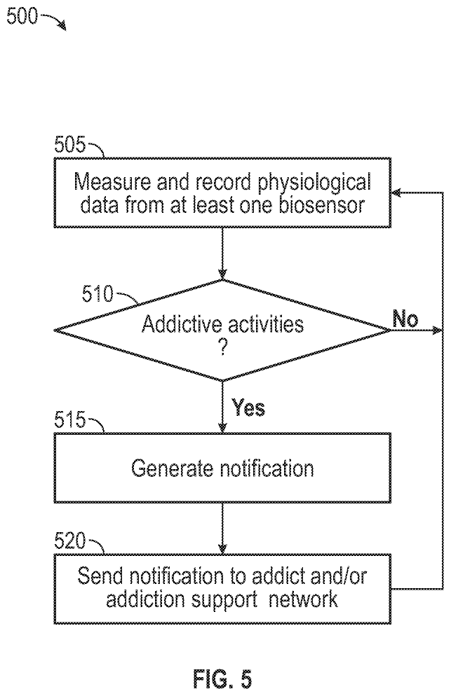

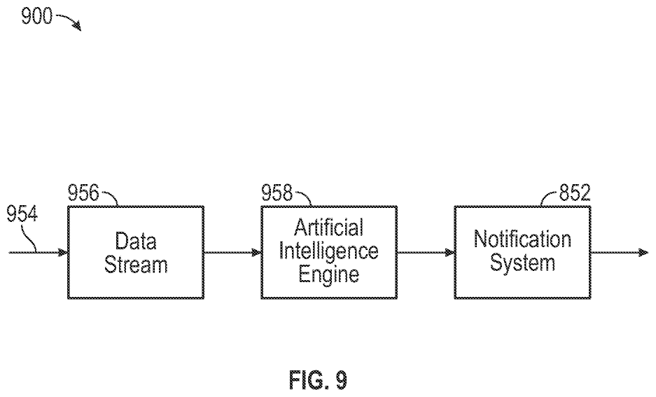

FIG. 5 is a flow diagram of a method 500 for monitoring and detecting one or more addictive activities of an addict, according to the present disclosure. The method 500 can include monitoring for one or more addictive activities of the addict and/or can include both monitoring and detecting the one or more addictive activities of the addict. The method 500 can be performed by the systems detailed herein, for example, the processor(s) of FIG. 9. In step 505, the method 500 includes measuring and recording physiological data from at least one biosensor. The at least one biosensor can include any of the biosensors in any of the wearable devices detailed herein.

In step 510, the method 500 includes determining if the addict has participated in addictive activities based on the physiological data. For example, the method 500 includes the processor receiving the physiological data from the at least one biosensor, and analyzing the physiological data (e.g., in real-time) and determining if the addict has participated in addictive activities. If the addict has not participated in addictive activities (step 510: No), the method 500 can continue to measure and record the physiological data from the at least one biosensor (e.g., the processor can continuously receive the physiological data).

In step 515, if the addict has participated in addictive activities (step 510: Yes), the method 500 includes generating a notification (e.g., an alarm or an alert) indicating that the addict has participated in addictive activities.

In step 520, the method 500 includes sending the notification (e.g., the alarm or the alert) to one or more devices of the addict and/or of an addiction support network of the addict. For example, the processor can send the notification via uni-directional or bi-directional communication.

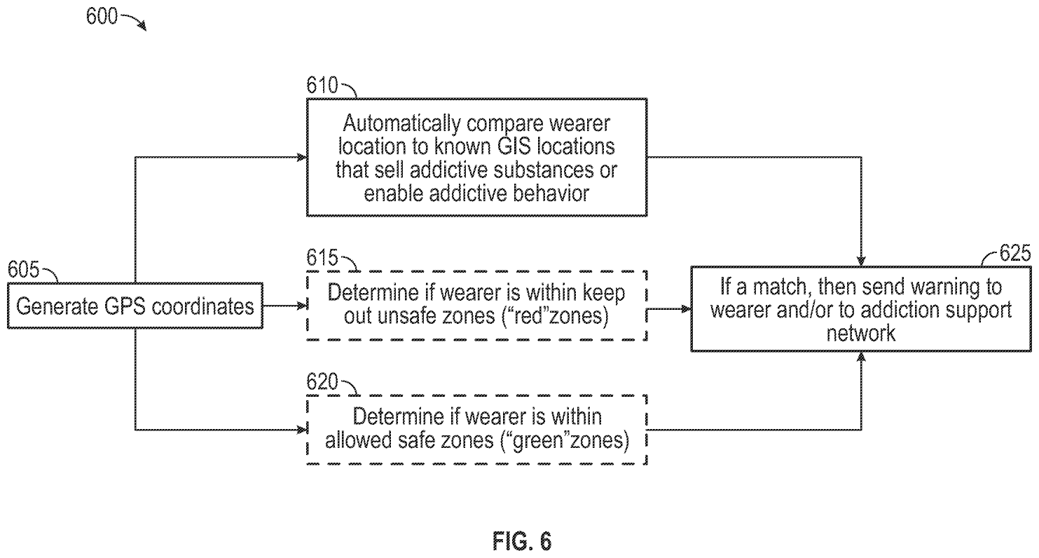

FIG. 6 is a flow diagram of a method 600 of monitoring and detecting one or more addictive activities of an addict, according to another embodiment. The method 600 can include monitoring for one or more addictive activities of the addict and/or can include both monitoring and detecting the one or more addictive activities of the addict. For example, FIG. 6 shows a schematic illustration of a high-level discussion of the geographical location and proximity-based location services and their use in providing alarms both to the wearer and to the wearer's addiction support network to modify the behavior of the wearer in real-time. The method 600 can be performed in combination with the method 500 of FIG. 5. One illustrative example of geographical location or proximity-based services is the use of a Global Position System (GPS) transceiver in a wearable or mobile device.

In step 605, the method 600 includes the GPS generating device GPS coordinates (e.g., in real-time). In step 610, the method 600 includes the server automatically comparing the wearer's locations to known geographic information system (GIS) locations that sell addictive substances (e.g., that an addictive alcoholic is within 50 feet of a restaurant, bar, package store, or sports venue which sells alcoholic beverages under state license) or that enable addictive behaviors. For example, the server receives the GPS coordinates from the GPS.

In some embodiments, the clinician supervising the use of the wearable device can (i) pre-define certain keep-out unsafe or danger zones, also referred to as “red” zones, geographically on a map or (ii) pre-define certain allowed safe zones, also referred to as “green” zones, geographically on the map. In step 615, the server determines if the wearer is within the keep out unsafe zones (“red” zones). In step 620, the server determines if the wearer is within the allowed safe zones (“green” zones).

In step 625, if a match occurs between the real-time location of the wearer and either known GIS locations, the keep out unsafe zones, or the allowed safe zones, the system warns the wearer (e.g., via alarms or notifications). Depending on the nature of the infraction, the system can automate communication with various members of the wearer's addiction support network and warn certain members of the wearer's addiction support network.

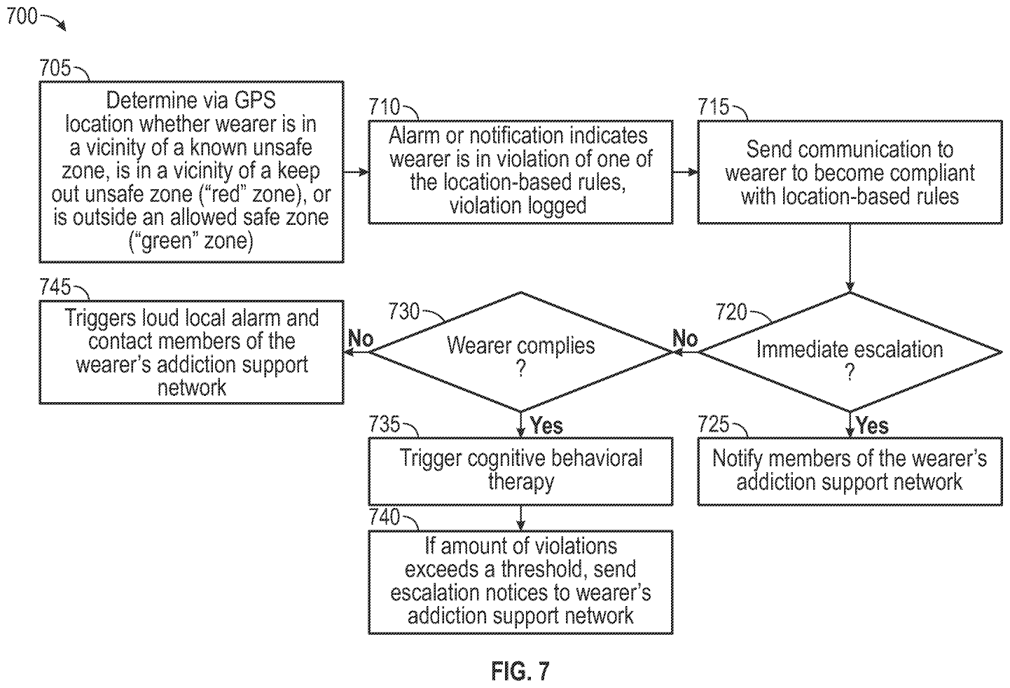

FIG. 7 is a flow diagram of a method 700 of monitoring and detecting one or more addictive activities of an addict, according to another embodiment. The method 700 can include monitoring for one or more addictive activities of the addict and/or can include both monitoring and detecting the one or more addictive activities of the addict. For example, FIG. 7 shows the details of the location-based subsystem and provides a lower-level discussion of the geographical location or proximity-based services and their use in alarms and modifying behavior. In particular, the system determines the wearer's physical location by the location services within the device worn by the wearer as described above. The system then compares (e.g., in real-time) the wearer's location or GPS coordinates with the three sets of locations described in FIG. 5, for example, i. the known GIS locations of relevance, ii. the pre-defined keep out unsafe zones (“red” zones), and iii. the pre-defined allowed safe zones (“green” zones), collectively referred to as “location-based rules.”

In step 705, the system determines, via the GPS location of the wearer, whether the wearer's location violates the location-based rules associated with the three sets of locations. For example, system determines whether the wearer is within a vicinity of a known unsafe zone based on the GIS location of relevance, is in the vicinity of a keep out unsafe zone (“red” zone), or is outside (e.g., too far outside) an allowed safe zone (“green” zone).

In step 710, if the wearer's location violates the rules associated with at least one of the three sets of locations, the system generates an alarm and/or a notification indicating a violation of one of the location-based rules and is logged in a recorded event log for later review and analysis.

In step 715, the system sends a communication to the wearer to alert the wearer of the location-based rules violation and requesting immediate compliance with the location-based rules by the wearer. The communication can include any form including, but not limited to, a message on the wearer's display interface, the vibration of a device, audible sound alarm pattern, phone call, video call, or the like.

In step 720, the system automatically determines whether an immediate escalation is needed based on a predefined set of criteria in the system. For example, if the addict is within two hundred yards of a keep out unsafe zone (“red” zone), then the system will only inform the wearer. In step 725, if the system determines an immediate escalation is needed (step 720: Yes) (e.g., detects the addict is less than thirty yards or within a keep out unsafe zone (“red” zone)), then the system will immediately notify members of the wearer's addiction support network. In this way, the members of the wearer's addiction support network can directly contact the wearer and attempt to lead to a behavior change that would bring the wearer into compliance with the location-based rules.

In step 730, if immediate escalation is not needed (step 720: No), the system continues to determine whether the wearer complies with the location-based rules. For example, the system monitors the wearer for behavior modification that would bring the wearer within compliance with the location-based rules.

In step 735, if the wearer does comply by moving to a geographic location that is compliant with their location-based rules (step 730: Yes), then the system triggers cognitive behavioral therapy to help the addict mitigate the addict's addictive urges and impulses. In step 740, if a number of violations exceeds a threshold, the system sends escalation notices to the wearer's addiction support network so that members of the wearer's addiction support network can help to mitigate the addict's urges and impulses.

In step 745, if the wearer does not comply (e.g., within a specified and communicated amount of time) by changing their geographical location (step 730: No), then the system triggers a loud local alarm on the wearer's device and contacts members of the wearer's addiction support network for immediate intervention in real-time.

In another embodiment, the definitions of safe zones (“green” zones) and unsafe zones (“red” zones) are defined in both space and time, as they are based on the individual subject's needs, and can change depending on the time of day since the safe zones and unsafe zones can be time-dependent and dynamic for both fixed and moving risks. For example, a restaurant during the day during the lunch hour could be a green zone, but after eight o'clock PM on any given evening, the restaurant becomes a red zone and is off-limits to a recovering alcoholic. Or in another example, highway rest areas where drugs are commonplace could be acceptable during the day when traveling to work but off-limits after six o'clock PM daily. Non-limiting examples of green zones could be the location of Alcoholics Anonymous meetings during the daily meeting time, school during the day, the wearer's apartment or home at night, and the school playground after school for adolescent wearers.

Moreover, in another embodiment, Bluetooth, Wi-Fi, or other two-way communication services could be employed to define a red zone proximally around other individuals like drug dealers or known relatives that engage in undesirable behaviors. Ultra-wideband technology and other location and proximity services are contemplated and considered part of the present disclosure.

In another embodiment, the analysis combines geographical location or proximity-based data with physiological adverse event data to notify first responders to intervene and improve health. Non-limiting illustrative examples include the determination that the wearer's respiration rate has dropped below seven breaths per minute or monitoring that their heart rate has fallen below forty beats per minute, which would trigger the notification system to call Emergency Medical Services (EMS) to the wearer's location provided by the geographical location sensor data to enable resuscitation and rescue of the addict in crisis.

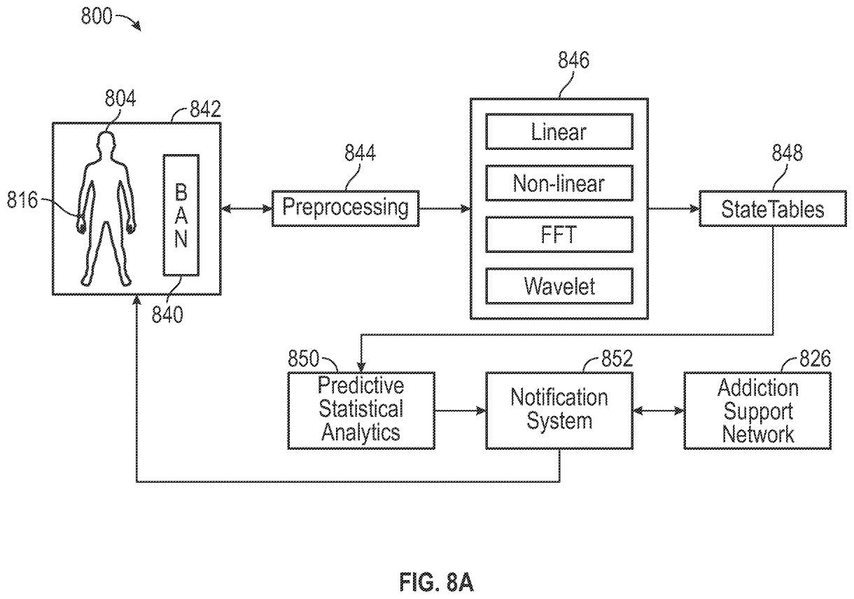

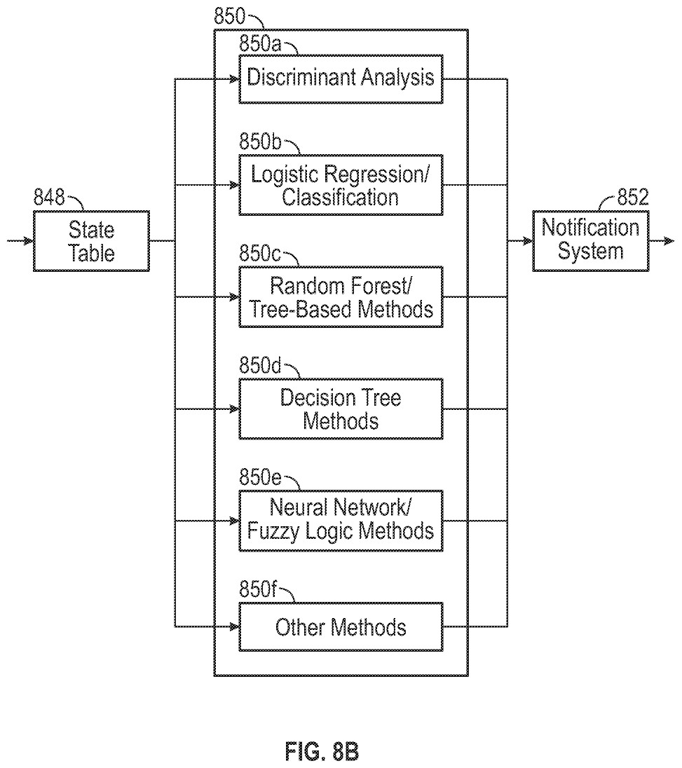

FIG. 8A is a schematic illustration of a data processing workflow 800 associated with signal preprocessing, data analysis, feature extraction, and predictive statistical modeling, according to the present disclosure. For example, for the system and methods of the present disclosure to work effectively, data collected from a wearable device placed on a subject patient must be processed using effective analytical tools. The data processing workflow 800 is described in FIG. 8 consists of a variety of data preprocessing, followed by data signal analysis, followed by feature extraction, and lastly the construction of predictive statistical models to either classify or regress a numerical output from the various input factors. The division between the local hardware (e.g., the wearable devices) and the cloud-based network (e.g., the cloud-based information technology infrastructure) can be drawn in multiple different locations and is arbitrary, but FIG. 8 presents the overall flow of information. Information starts from a wearable device 816 placed on an addict 804 where the wearable device 816 is part of a body area network (BAN) 840 that is inside a personal area network (PAN) 842. For example, the biosensors of the wearable device 816 generate biosensor data. The information (e.g., raw biosensor data) is streamed from the PAN 842 into a preprocessing module 844 that flags artifacts and verifies the integrity of the various streams of biosensor data that have been acquired from the wearable device 816 on the addict 804 in the PAN 842.

Once the data has been preprocessed and data streams have been verified for integrity, the data streams with integrity are then sent to a data analysis toolbox 846 which consists of several different signal processing and analytical methods and tools that can each be applied in parallel to the preprocessed data. For example, nonlimiting signal processing and data analysis methods include linear analysis, non-linear analysis, spectral Fast Fourier Transform (FFT) analysis, and multispectral wavelet transform analysis. At the back end of the data analysis toolbox 846, features are extracted from each of the various data analytical analyses and are sent to one or more state tables 848 from which predictive statistical analysis is undertaken by a predictive statistical analytics module 850 using the extracted features as predictive model input variables. The output of the statistical predictive analysis can either be i) the classification of a subject into a state or group or class such as “normal,” “under the influence of opioids,” “agitated,” etc., or ii) the regression to a continuous variable numerical score from the set of extracted feature input variables. This is more often used when trying to assess the probability of a diagnostic state or a prognostic condition that may or may not occur sometime in the future. The results of the predictive statistical analysis by the predictive statistical analytics module 850 are then pushed to a notification system 852, also referred to as a communication system, that is then bidirectionally connected to an addiction support network 826. As detailed above, the addiction support network 826 can include family and friends of the subject, recovery management personnel, clinicians of a crisis or noncrisis variety, emergency medical technicians who engage in crisis management, physicians a) in the emergency department engaged in a crisis or b) in an office setting engaged in non-crisis healthcare. The notification system 852 can provide input back to the PAN 842 to inform others including the subject of the results from the data processing workflow consisting of preprocessing, data analysis, feature extraction, and predictive statistical modeling.

In addition, the notification system 852 can disable machinery in the wearer's local environment, such that the addict 804 is prevented from starting their car if the addict 804 has alcohol on their breath. The system can determine the addict's fitness for duty such as driving a truck through the night or operating a bulldozer. The system can further make the proper notifications to enable or disable access to a building if the addict 804 has recently participated in addictive activities (e.g., engaged in the consumption of addictive substances or participated in addictive behaviors).

FIG. 8B is detailed view of the predictive statistical analytics module 850, according to the present disclosure. The state table(s) 848 are populated with extracted data features can be subjected to model building from several different classes of models. Examples of statistical learning models is provided in Hastie, Tibshirani, and Friedman, The Elements of Statistical Learning: Data Mining, Inference, and Prediction. Springer 2 nd Ed. 2009.

The predictive statistical analytics module 850 can include discriminant analysis methods 850a, logistic regression/classification methods 850b, random forest/tree-based methods 850c, decision tree methods 850d, neural network/fuzzy logic methods 850e, and/or other methods 850f. The discriminant analysis methods 850a include either linear or quadratic discriminant analysis, but may include other types of discriminant analysis, to create models to classify or regress the input variables. Models are typically developed or “trained” on one data set, verified on an independent second data set, and validated with no more changes to the model on a third independent data set. Often, the validation data are collected prospectively in time to further enhance the performance evaluation of the model. When necessary, internal cross-validation, leave-one-out, and other techniques can be employed validate the data with minimal data sets. The logistic regression/classification methods 850b can include either logistic regression to a number or logistic classification to a group or class, and are often built to develop the most accurate predictive models. The random forest/tree-based methods 850c include constructing a multitude of decision trees when the model is trained and are very effective when applied to certain types of data sets. The decision tree methods 850d are typically based on a few parameters when well-defined inputs are available. Neural networks/fuzzy logic methods 850e are also highly effective as predictive models where an in-silico attempt is made to model the brain with layers of neurons with various synaptic connection types and weights. Other methods 850f are equally contemplated in the present invention, such as, for example, support vector machine and machine learning.

FIG. 9 is a schematic illustration of a data processing workflow 900, according to another embodiment. The data processing workflow 900 includes biosensor data that creates a data stream that can be analyzed by an artificial intelligence engine before triggering a notification system. For example, the data processing workflow 900 includes the use of an artificial intelligence engine after a data stream has been created and upstream of the notification system. FIG. 9 shows various biosensor outputs 954 that collectively constitute a data stream 956. The data stream 956 is then fed into an artificial intelligence engine 958 where various signal conditioning tasks can take place, various signal processing tasks can occur, including feature extraction, and then various artificial intelligence approaches can be leveraged to find the best signatures that the notification system 852 should be triggered and either the addict 804 (FIG. 8A) themselves notified or possibly certain members of the addict's addiction support network 826 (FIG. 8A).

In one embodiment, the system can make a statistical comparison to baseline measurements conducted on the wearer and determine that the system is no longer being worn by the addict wearer. The signature of the wearer is precise enough that the system can distinguish the wearer of interest from an imposter and determine the system is not being worn by the proper person to prevent fraud on the system.

As one can see from the above examples communication amongst the various users is essential to the successful outcome for the subject (e.g., the addict). It is vitally important to understand how the users and the members of the subject's addiction support network communicate to effectively achieve the positive outcomes desired, i.e., less addictive substance abuse and less addictive behaviors.

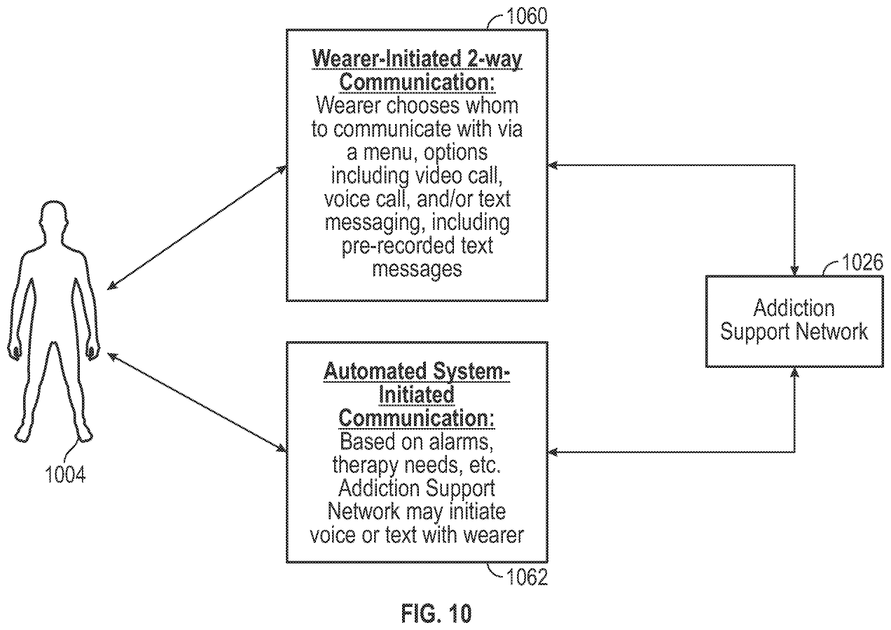

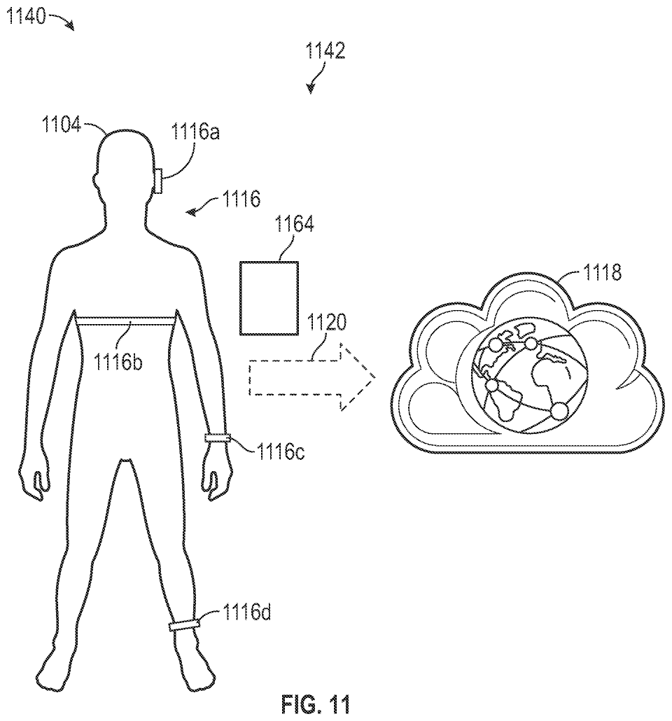

FIG. 10 is a schematic illustration of two primary modes of communication between an addict 1004 and an addiction support network 1026 for the addict 1004, according to the present disclosure. In block 1060, the addict 1004 can willfully initiate communication (e.g., wearer-initiated 2-way communication) via a menu of options including, but not limited to, video calls, voice calls, text messaging, tweets, and pre-recorded messaging. Other modes of communication that are being developed are contemplated as inclusive of the present invention as well. Additionally, or alternatively, in block 1062, the system can automatically initiate communication (e.g., automated communication) for the wearer based on the triggering of various types of alarm conditions that can derive from the various biosensors worn by the subject, or the location-based rules predefined for the wearer as described earlier. The wearer-initiated communication (e.g., block 1060) or the automated system-initiated communications (e.g., block 1062) take place with the various members of the addiction support network 1026.

In another embodiment, the system can decide if the subject is engaged in secondary support associated activities. The secondary support associated activities can include, but are not limited to, using a credit card at a shady establishment or withdrawing cash from an ATM in the vicinity of a known risk establishment or at an at-risk time of the day (e.g., such as late evening).

FIG. 11 is a schematic illustration of a personal area network (PAN) 1142, according to the present disclosure. FIG. 11 shows the sensors proximal to the addict, the body area network, and the information connection to the cloud-based network (e.g., the cloud-based information technology stack). The data needs to flow cleanly from the device which consists of multiple biosensors that are worn by the subject or wearer to the cloud-based network. For this to occur, a body area network (BAN) 1140 is created on an addict 1104. For example, the addict 1104 wears one or more wearable devices 1116 on one or more locations on the addict's body. The one or more wearable devices 1116 include one or more sensors (e.g., biosensors) therein. For example, the one or more wearable devices 1116 include a first wearable device 1116a that is placed on an ear of the addict 1104, a second wearable device 1116b that is placed on the chest of the addict 1104, a third wearable device 1116c that is placed on an arm, a wrist, or a finger of the addict 1104, and a fourth wearable device 1116d that is placed on a leg, a knee, an ankle, a foot, or a toe of the addict 1104. In one embodiment, the third wearable device 1116c is referred to as a main module and includes a display interface for the addict 1104 to view information (e.g., alerts, notifications, unsafe zones, safe zones, etc.), much like a smartwatch from Apple®, Samsung®, Garmin®, or any other manufacturer. The personal area network 1142 also includes one or more proximal sensors 1164. The proximal sensors 1164 are sensors in the proximity of the wearer but not on the wearer, such as, for example, an infrared motion sensor, a room-temperature sensor, a smoke sensor in the ceiling, or the like. The proximal sensors 1164 form a part of the PAN 1142 along with the BAN 1140 as a subset of the PAN 1142. The data from all sensors (e.g., from the biosensors of the one or more wearable devices 1116 and/or from the proximal sensors 1164) is moved securely and privately through a data pipeline (as indicated by arrow 1120) to a cloud-based network 1118 (e.g., a cloud information technology infrastructure).

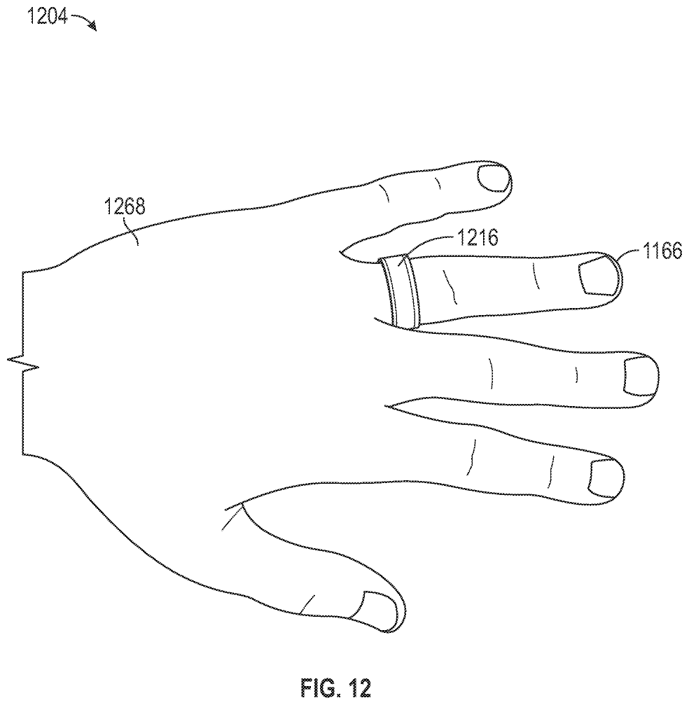

FIG. 12 is a schematic illustration of one or more ring-based wearable devices 1216, according to the present disclosure. The ring-based wearable devices 1216 include one or more biosensors and can be placed on the addict's fingers or toes. In the embodiment of FIG. 12, the ring-based wearable devices 1216 are worn around a finger 1166 or fingers of a hand 1268 of an addict 1104, whereby the ring-based wearable devices 1216 comprise the various biosensors within its ring-shaped form factor. The ring-based wearable devices 1216 enable a non-invasive, continuous measuring of important physiological variables, such as heart rate, respiratory rate, skin temperature, and galvanic response. Additionally, or alternatively, one or more of the ring-based wearable devices 1216 can be worn on one or more toes on a foot of the addict 1204.

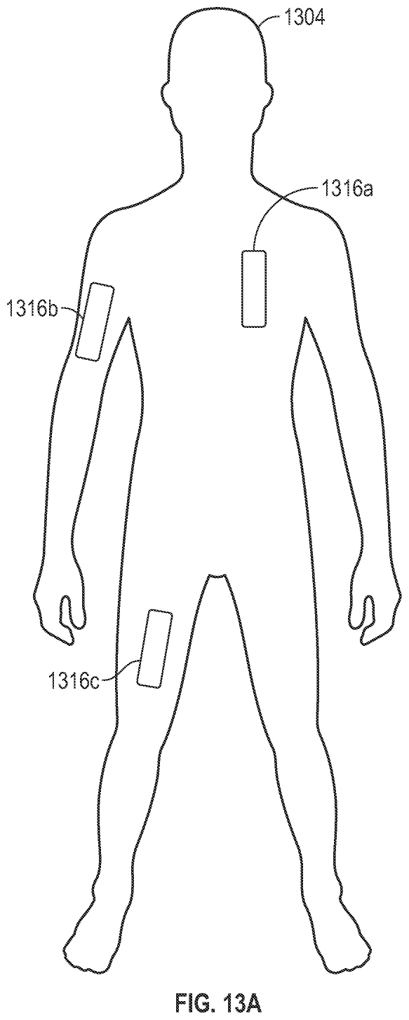

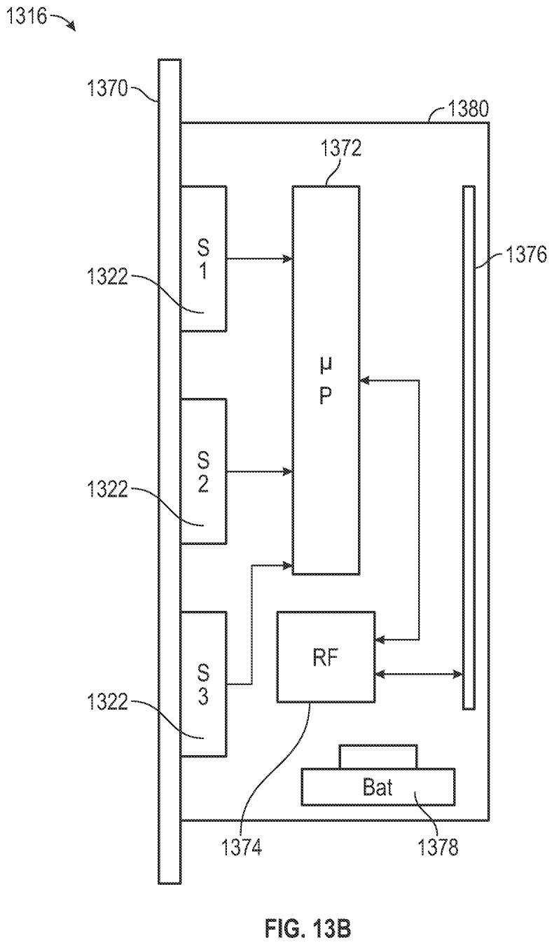

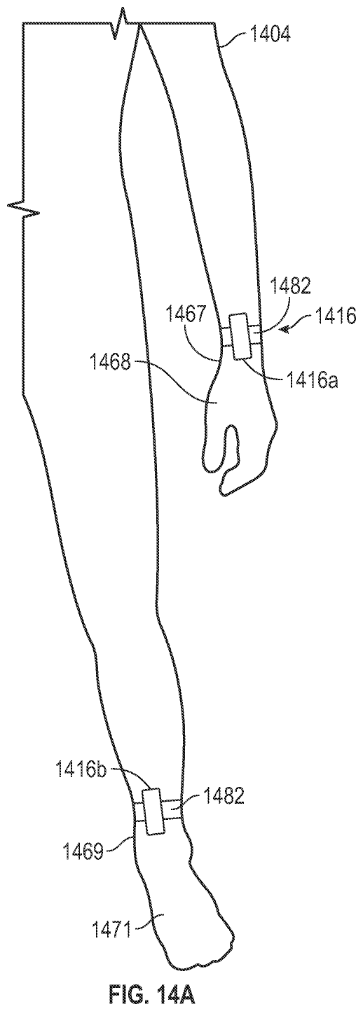

FIG. 13A is a schematic illustration of one or more adhesive-based wearable devices 1316, according to the present disclosure. The adhesive-based wearable devices 1316 include one or more biosensors and can be placed on the addict's skin along the thorax, arms, legs, neck, or head. For example, an addict 1304 can wear one or more adhesive-based wearable devices 1316 that are adhesively mounted to the addict's skin. The one or more adhesive-based wearable devices 1316 can include, for example, a first adhesive-based wearable device 1316a attached to a chest of the addict 1304, a second adhesive-based wearable device 1316b attached to an arm of the addict 1304, and/or a third adhesive-based wearable device 1316c attached to a leg of the addict 1304. The adhesive-based wearable devices 1316 can each be colloidally or adhesively mounted in the form factor of a patch that contains biosensors, communications, etc. as detailed below with respect to FIG. 13B.

FIG. 13B is a schematic illustration of an adhesive-based wearable device 1316, according to the present disclosure. For example, FIG. 13B shows the details of the adhesive-mounted biosensors and communications equipment. The adhesive-based wearable device 1316 includes an adhesive layer 1370 that adheres to the addict's skin. The adhesive-based wearable device 1316 also includes one or more biosensors 1322 (e.g., labeled S1, S2, S3, etc.) that send data streams to a microprocessor 1372 that collects and time-stamps all the various data streams from the one or more biosensors 1322 with microsecond precision (e.g., or more) so that various data streams are registered in time across all the biosensors. The output from the microprocessor 1372 is sent to a radio 1374 (e.g., radio frequency) that includes, for example, WiFi, Bluetooth, ANT, or any other wireless protocol or technology. An antenna 1376 transmits and receives data from the personal area network (e.g., any of the personal area networks detailed herein) that is connected to the cloud-based network. The adhesive-based wearable device 1316 also includes a battery 1378 that powers the adhesive-based wearable device 1316. The adhesive-based wearable device 1316 includes a case 1380 that encases the one or more biosensors 1322, the microprocessor 1372, the radio 1374, the antenna 1376, and the battery 1378. Further, systems that use two biosensors, three biosensors, four biosensors, five biosensors, six biosensors, and even more are contemplated and a part of the present disclosure.

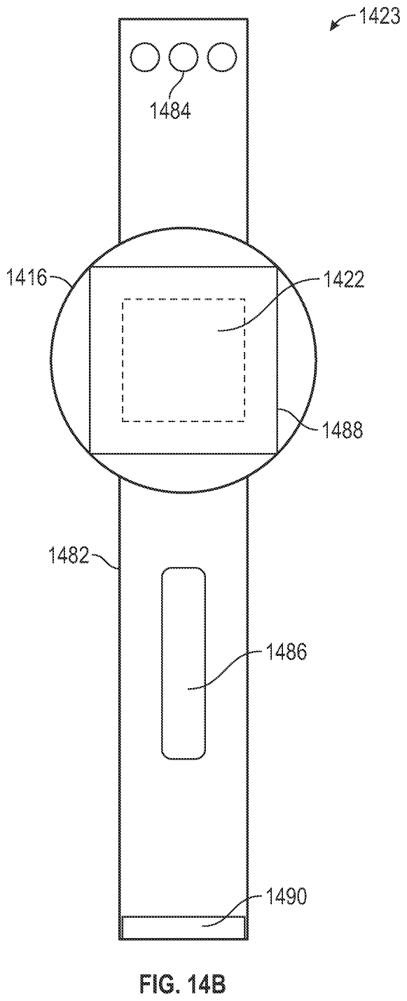

FIG. 14A is a schematic illustration of one or more band-mounted wearable devices 1416, according to the present disclosure. For example, the band-mounted wearable devices 1416 are worn along the arms and legs of an addict 1404. The one or more band-mounted wearable devices 1416 are held in place by a band 1482 of fabric, sheet, mesh, or other material that can support the band-mounted wearable device against the addict's skin. For example, the one or more band-mounted wearable devices 1416 include a first band-mounted wearable device 1416a wearable on a wrist 1467 by a hand 1468 of the addict 1404 and/or a second band-mounted wearable device 1416b wearable on an ankle 1469 by a foot 1471 of the addict 1404. This enables the addict to have the best place for communications on the wrist. Such a configuration is a step up in fidelity of signals. The ear lobe is another high-fidelity location to be used for such a purpose. Moreover, having the band-mounted wearable device 1416 in a watch-like position along the wrist 1467 of the addict 1404 enables technological smartwatches and other display-like devices to not only collect biosensor data, but also provide a variety of feedback signals to the addict 1404, including, but not limited to, inbound voice phone calls, inbound text messages, and inbound email, among other signals and communication.

FIG. 14B is a detailed schematic illustration showing of a band-mounted wearable device 1416, according to the present disclosure. The band-mounted wearable device 1416 is supported by the band 1482 that can be wrapped around a portion of the body of the addict. More particularly, the band-mounted wearable device 1416 includes a sensor array 1423 including one or more biosensors 1422. As detailed above, the one or more biosensors can include, but is not limited to, PPG, EDA, temperature, and transdermal secretions. The sensor array 1423 can also include electrodes 1484 and other sensors 1486, that can include, but are not limited to, chemical sensors, acoustic sensors, electromagnetic sensors, and/or biological sensors. The sensors of the sensor array 1423 are most typically mounted in a substrate made of fabric, but neoprene, silicone, plastic, or any other matter that will support the one or more biosensors 1422 and other sensors (e.g., the electrodes 1484 and the other sensors 1486) on the surface of the addict's body could suitably work as well. The band-mounted wearable device 1416 also includes a microprocessor-controlled display 1488, such as, for example, an LCD, an LED, or other two-dimensional display devices, so that messages that include graphical information can be displayed for the addict, and the addict can view the messages similar to a smartwatch. The band-mounted wearable device 1416 also includes a closure device 1490, such as, for example, Velcro®, clasp, or any other type of closure device, secures the band 1482 around a part of the body, typically, the wrist or the ankle of the addict. The band-mounted wearable device 1416 can also include an internal speaker that can audibly play aloud digital messages using text-to-voice technology, or other forms of audible alerts. Smartwatches from Apple®, Samsung®, Garmin®, or the like, are fully contemplated as suitable starting devices for a sensor array expansion to include other types of biosensors as described herein.

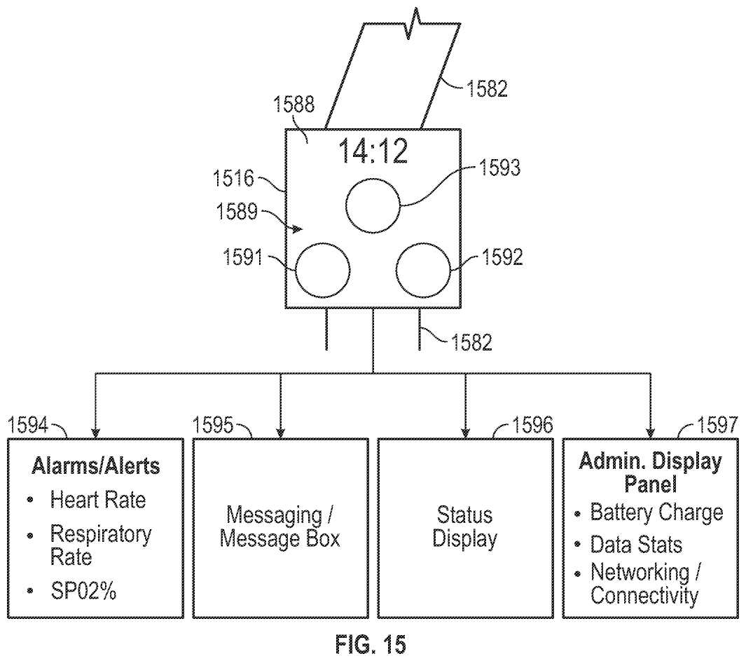

FIG. 15 is a detailed schematic illustration showing a band-mounted wearable device 1516 having a mobile-based software application, according to the present disclosure. While the embodiment of FIG. 15 details a band-mounted wearable device 1516, the software application detailed herein can be used on a mobile device or other computing device, such as, for example, a smartphone, a personal computer, a laptop, smart glasses, or the like. The band-mounted wearable device 1516 includes a band 1582 holding the band-mounted wearable device 1516 securely to the addict's skin, a display 1588 that displays not only the time, but also aspects of one or more software applications 1589 that include a panic button 1591, a communication button 1592, and a menu button 1593. FIG. 15 shows one configuration of the panic button 1591, the communication button 1592, and the menu button 1593, but the display 1588 can display any configurations of such buttons. The one or more software applications 1589 also include a set of alarms or alerts 1594 that include, for example, a heart rate respiratory rate or other biosignals that can be tailored to a wearer to signal adverse situations or behaviors (e.g., addictive activities). For example, when an addict that is addicted to alcohol is near a bar serving alcohol at a sports stadium the addict's heart rate may begin to elevate, and the addict's breathing may get shallow such that the addict's respiratory rate may increase significantly as well. The monitoring by the band-mounted wearable device 1516 can then send an alert to the addict on the display 1588 that the addict's heart rate and respiratory rate are significantly altered and the addict is likely in the presence of an alcohol opportunity. The one or more software applications 1589 can then instruct (on the display 1588) the addict to move away from the source of alcohol to reduce or eliminate the temptation to deviate in addictive substance or addictive behavior. The set of alarms or alerts 1594 can also alert the addict of blood oxygen level (e.g., oxygen saturation, SpO2%), and/or any other indicators of the addict participating in addictive activities.

The one or more software applications 1589 also include a messaging application 1595 that includes at least an inbox and an outbox that allow the addict to both receive and transmit text style messages with or without attachments, videos, or other forms of data, both to and from clinicians and healthcare providers that are caring for the addict, family members of the addict, and/or friends of the addict. For example, the system enables communication with the addiction support network of the addict to provide support and intervention as necessary.

The one or more software applications 1589 also include a status display 1596 that displays an assortment of information to the addict on the display 1588. Nonlimiting examples include, but are not limited to, 1) the location of emergency medical support (EMS) that may be dispatched to support the addict in crisis, 2) time since last consumption of an addictive substance, or 3) time since last participation in addictive behavior, or 4) the addict's present mental capacity based on the physiologically measured variables and embedded algorithms of the present invention, e.g., if the addict was inebriated from alcohol or high on marijuana, etc.

The one or more software applications 1589 also include an administrative display panel 1597 that indicates device state of the band-mounted wearable device 1516, such as, for example, battery charge, data stored on the device or space still available, how much data has been transported from the device to the cloud-based network, how many messages have been sent and received in the last day, week, month, quarter, or year, as nonlimiting examples. The administrative display panel 1597 can also include a settings subpanel that enables the addict to select metric or imperial units including Celsius and Fahrenheit temperature scales as well as other personalization and customization options.

The one or more software applications 1589 can include separate applications as part of a device operating system, and/or can be combined into a single software application, or into groups of software applications.

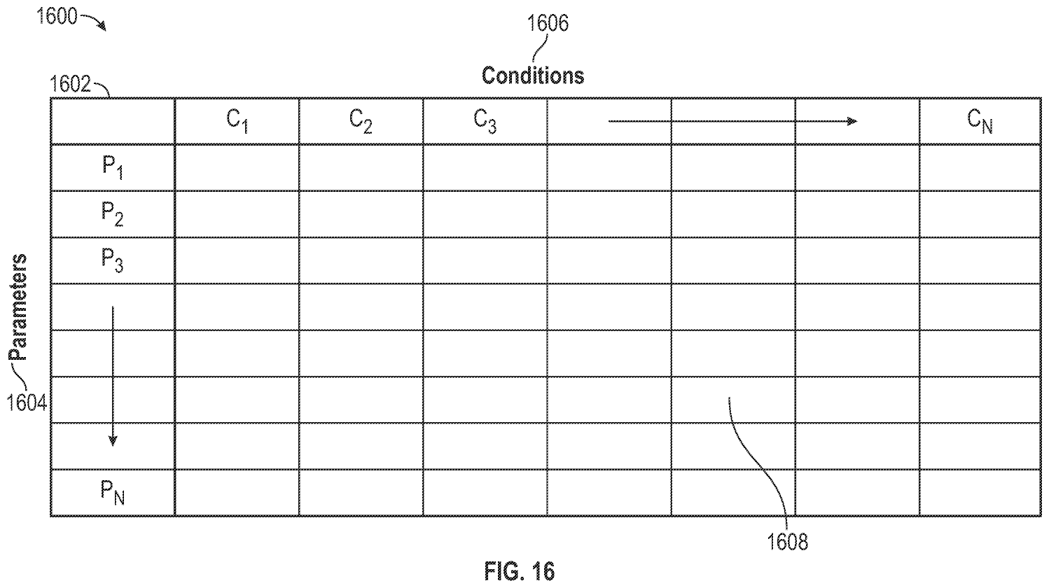

FIG. 16 is an illustrative table 1600 showing a generic state engine with various output conditions (as columns) and various biosensor-derived measurement parameters as input variables (as rows), according to the present disclosure. In one embodiment of the present disclosure, the system through the various hardware and software systems detailed herein collects biosignal data from the addict, extracts an ensemble of parameters P₁ through P_{N}, and can then determine the condition or state of the wearer physically, mentally, and/or emotionally. Such a process is illustrated in FIG. 16 in a generic state engine 1602 (e.g., any of the state engines detailed herein) whereby various parameters 1604 P₁ through P_{N} are listed as rows and various physical, mental, and/or emotional states or conditions 1606 are listed as columns C₁ through CN. As a non-limiting example, parameter P₁ is heart rate (HR), parameter P₂ is respiratory rate (RR), parameter P₃ is core temperature (T_{core}), parameter P₄ is saturated oxygen (SO₂), parameter P₅ is secreted alcohol concentration, parameter P₆ is secreted opioid concentration, parameter P₇ is secreted cannabinoid, and parameter Pg is accelerometer variance (sigma\hat{ }2) summed over all three axes (X, Y, Z) during a 30-second postural challenge. The measured and calculated parameters 1604 are the rows of the state engine 1602. Then, by measuring the parameters 1604 in the various conditions 1606, the system generates a predictive statistical model or signature of each condition 1606 based on the input parameters 1604. A statistical predictive model, of those described in Hastie et al, 2009, is designed to take input features as inputs and either classify an unknown set of features into a class, state, or condition as an output. Predictive models can also be created and utilized in a regression mode whereby input features can be used to calculate a continuously variable output index which is then used quantitatively to characterize the subject, or their state or condition. Each cell 1608 of the table 1600 represents a respective parameter 1604 for a respective condition 1606.

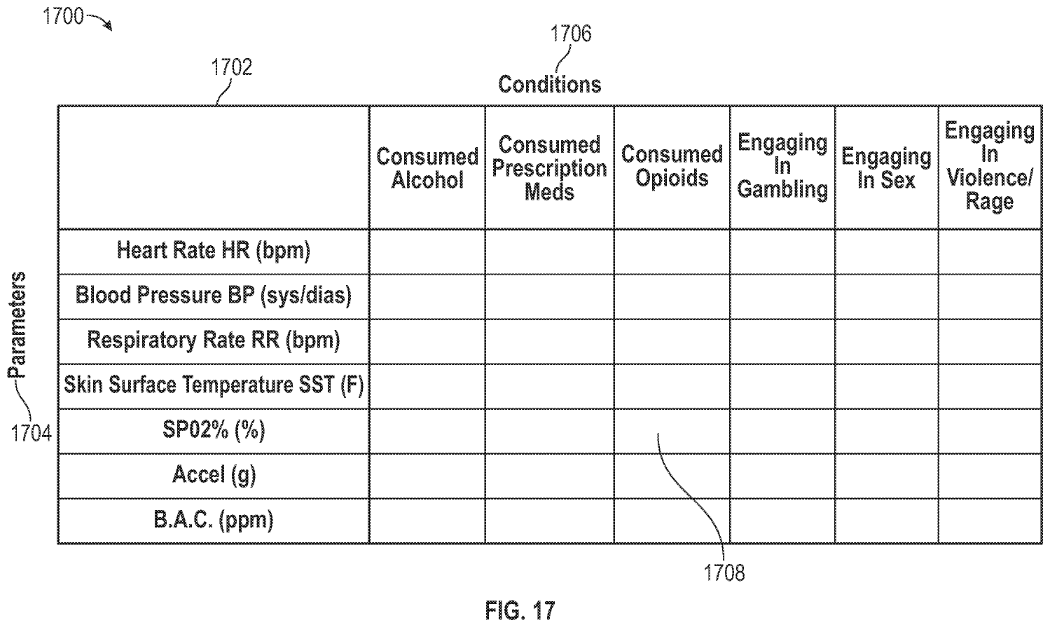

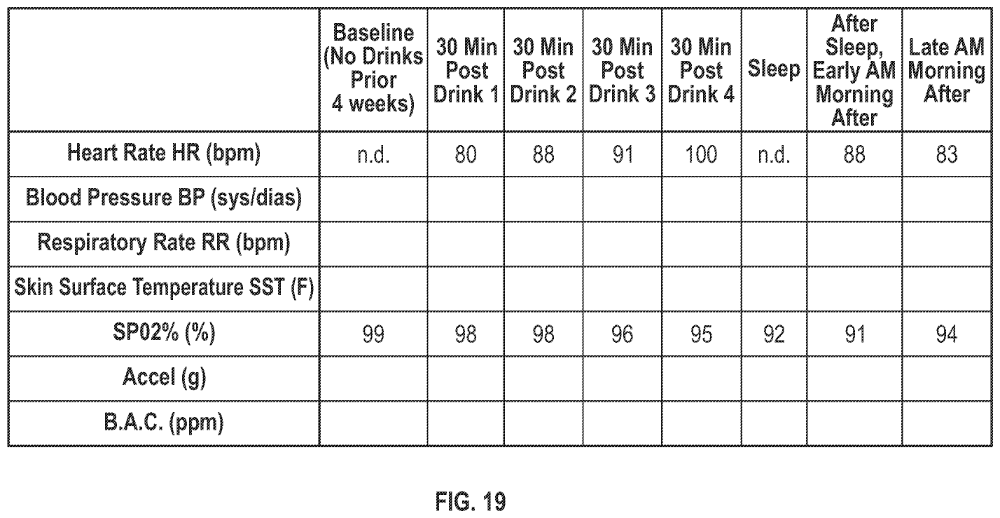

FIG. 17 is a table 1700 showing a state engine 1702 for six exemplary conditions, showing addiction conditions as columns and various biosensor-derived measurements as input variables as rows, according to the present disclosure. For example, FIG. 17 is an example of a high-level state engine for various addictive conditions, some substance-based, some behavior-based. FIG. 19 shows the state engine 1702 with a list of parameters 1704 (as rows) and various conditions 1706 (as columns). Down the left column the list of parameters 1704 is delineated whereby the first parameter is heart rate (HR) in beats per minute (bpm), then blood pressure (BP) (systolic/diastolic), then respiratory rate (RR) in bpm, then skin surface temperature (in degrees Fahrenheit), saturated oxygen percent SPO2(%), 3-axis combined accelerometry (g), and lastly, Blood Alcohol Concentration (BAC) (in parts per million or ppm). The list of parameters 1704 can then be used to deduce various conditions 1706 listed as columns across the state engine 1702. In the first column, “consumed alcohol” is the first condition, the next column is “consumed prescription medicine,” the next column is “consumed opioids,” the next column is “engaging in gambling,” the next column is excessive “engaging in sex,” and the last column is excessive “engaging in violence/rage.” In its simplest form, the state engine 1702 classifies a subject's condition based on a set of parameter values measured from devices worn by the subject (e.g., the wearable devices detailed herein). Each cell of the table 1700 represents a respective parameter 1704 for a respective condition 1706. For example, cell 1708 shows the SPO2% parameter for the “consumed opioids” condition.

In one embodiment, the device may automatically trigger injection of NARCAN or other appropriate pharmaceutical agents to reduce or minimize the impact of a possible overdose of an addictive substance. In another embodiment, the device may interface with an automobile, airplane, boat, train, construction equipment, or other machinery to communicate the wearer's suitability for operating such potentially dangerous equipment.

While the above description contains many specifics, these specifics should not be construed as limitations on the scope of the invention, but merely as exemplifications of the disclosed embodiments. Those skilled in the art will envision many other possible variations that are within the scope of the invention. The following examples will be helpful to enable one skilled in the art to make, use, and practice the invention.

### EXAMPLES

### Example 1: Study of Alcohol on Physiological Parameters

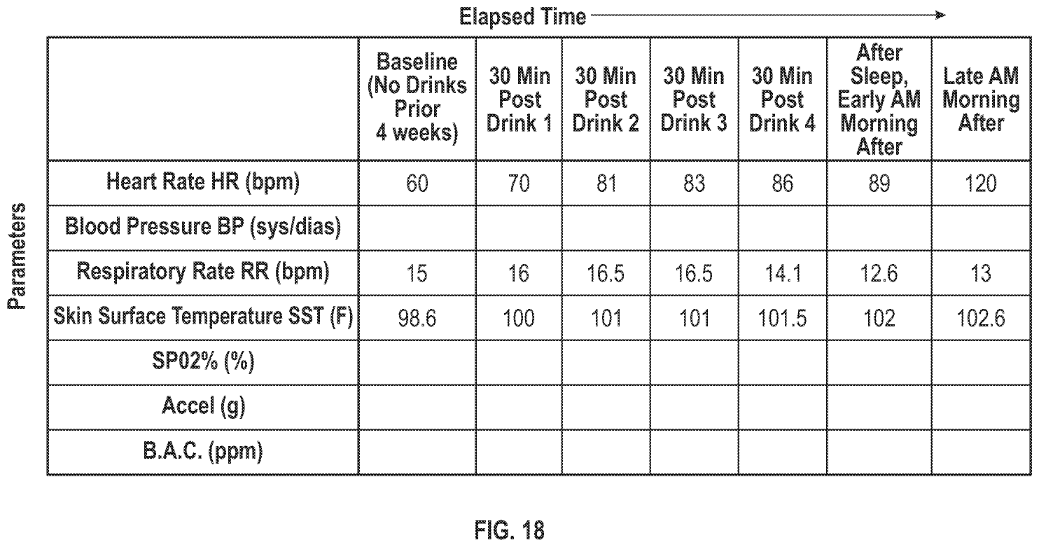

FIG. 18 is a table of biosensor-derived measurement parameters (as rows) collected over time after intervals of alcohol consumption (as columns, moving from left to right in time) for a single individual (N=1). Individual subjects were enrolled in an informal study to evaluate the physiological parameters available at the time. FIG. 18 is an example of a time course of several hours for a single individual that was consuming alcohol. Time runs from left to right while various parameters are listed in each row. FIG. 18 shows heart rate HR measured in beats per minute in the top row, followed by Blood Pressure BP measured as systolic over diastolic, then Respiratory Rate RR measured in breathes per minute, then Skin Surface Temperature SST measured in degrees Fahrenheit (F), then Saturated Partial Oxygen percentage SPO2% measured in the percentage from zero to 100%, then accelerometer-based stability measured in units of the reference for earth's gravity acceleration g (either 9.8 m/s² or 32 ft/s²), with Blood Alcohol Concentration (B.A.C.) as measured in parts per million (ppm) in the last row of the table.

The experimental protocol consisted of taking several measurements as a baseline before the subject consumed any alcohol (first column), followed by the same measurements thirty minutes after consuming one drink, followed in the next column by the same measurements thirty minutes after consuming a second drink which was administered thirty minutes after consumption of the first drink. A third drink was consumed sixty minutes after finishing the second drink and the measurements were made thirty minutes after finishing the third drink and are shown in the next column. The next column shows the data that was measured thirty minutes post drink four which was consumed sixty minutes after finishing drink three. After the fourth drink, the subject was allowed to sleep for eight hours and the early morning data after rest that was measured is shown in the next column, followed by the data that was measured in the late morning in the last column to the right. As time moves horizontally from left to right, this can help define a predictive signature for someone under the influence of alcohol. FIG. 18 shows an elevated heart rate HR and skin surface temperature SST due to the consumption of alcohol in this subject. The respiratory rate decreased over time while consuming alcohol and then recovered once the substance was washed out of the body.

FIG. 19 is a table of biosensor-derived measurement parameters (as rows) collected over time after intervals of alcohol consumption (as columns, moving from left to right in time) for a second individual (N=1). FIG. 19 shows the same time course of data as FIG. 18, but collected from a second individual on a different day. In this study of the same consumption protocol, both Heart Rate HR and Saturated Partial Oxygen Percentage SPO2% measurements are shown which were recording during the alcohol consumption protocol. Again, an elevated Heart Rate HR is shown during consumption of alcohol in addition to a reduction in the SPO2% during alcohol consumption.

### Example 2: Second Study of Alcohol on Physiological Parameters

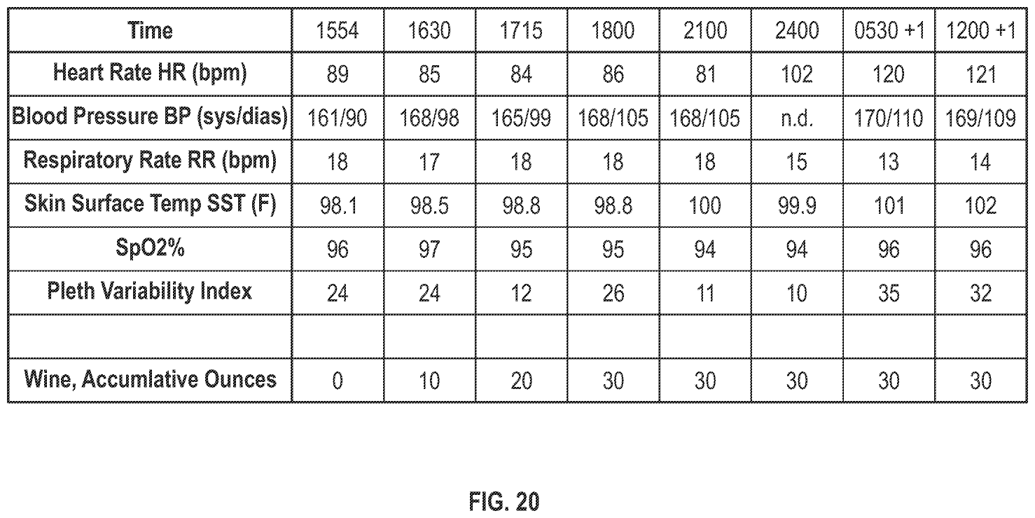

FIG. 20 is a table of biosensor-derived measurement parameters (as rows) collected over time after intervals of alcohol consumption (as columns, moving from left to right in time) for a third individual (N=1). Similar to Example 1, an individual was informally enrolled to consume three glasses of wine over time and both the time of day and biosensor data were recorded. FIG. 22 is an example of a time course of several hours for a single individual who was consuming wine, with the total amount of wine consumed in ounces recorded along the bottom. Time runs from left to right in the top row using 24-hour military time notation, while various parameters are listed in each row. In this example, heart rate HR is measured in beats per minute in the top row, followed by Blood Pressure BP measured as systolic over diastolic, then Respiratory Rate RR measured in breathes per minute, then Skin Surface Temperature SST measured in degrees Fahrenheit (F), then Saturated Partial Oxygen percentage SPO2% measured in the percentage from zero to 100%, then the Pleth Variability index (PVi), a measure of the variability of the PPG respiratory rate over time). PVi provides a continuous noninvasive measure of the relative variability in the photoplethysmography (pleth) amplitudes during respiratory cycles that may be used as a dynamic indicator of fluid responsiveness. As such, PVi may be used as an indicator for the degree of hydration or dehydration of the wearer. Importantly, PVi may be used to predict hypotension induced by drugs and activities. hypotension due to Elle vasodilator effects of drugs may cause decreased critical organ perfusion and increased cardiac and cerebral complications, and lastly, the total amount of wine consumed in ounces along the bottom row of the table.

The experimental protocol consisted of taking measurements as a baseline before the subject consumed a 10-ounce glass of wine (first column at time 1554), followed by the same measurements thirty minutes after consuming one glass of wine (1630), followed in the next column (1715) by the same measurements thirty minutes after consuming the second glass of wine which was administered immediately after the measurements at 1630. The third glass of wine was consumed after finishing the second drink measurements at 1715 and the measurements after the third glass of wine were made at 1800 and are shown in the next column. The next column shows the data that was measured three hours later at 2100 (or 9:00 PM), while the next column shows the measurements made at 2400 (midnight) three hours later. The subject was allowed to sleep for five and one-half hours until 0530+1 the next day when the measurements were recorded again. Lastly in the far-right column, the measurements at 1200+1 (noon the day after drinking) are shown. As time moves horizontally from left to right, this can help define a predictive signature for someone under the influence of alcohol. FIG. 20 shows an elevated heart rate HR and skin surface temperature SST due to the consumption of alcohol in this subject.

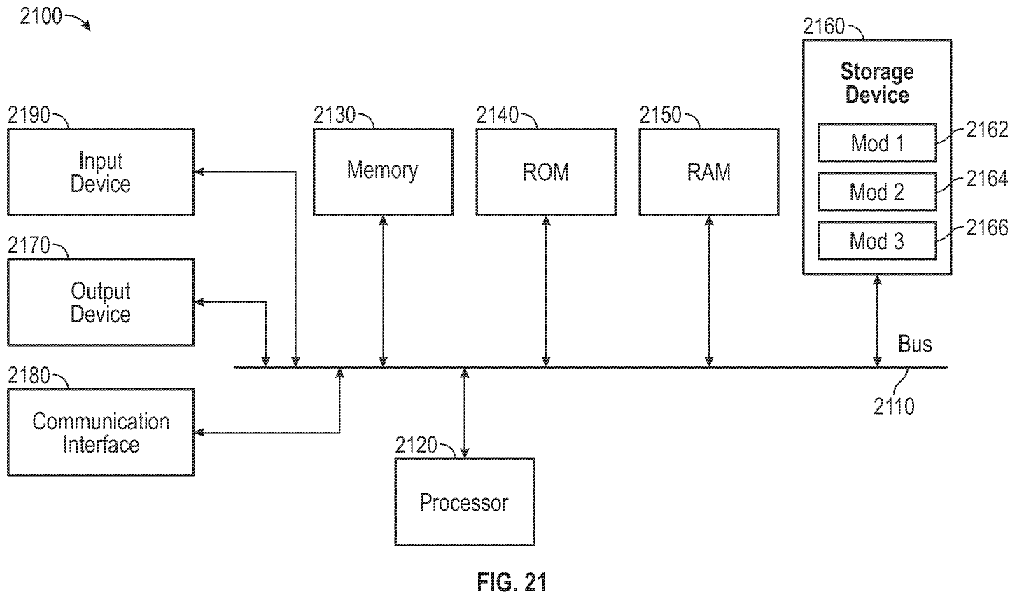

FIG. 21 illustrates an exemplary system that includes a general-purpose computing device 2100, including a processing unit (CPU or processor) 2120 and a system bus 2110 that couples various system components including a memory 2130 such as read-only memory (ROM) 2140 and random-access memory (RAM) 2150 to the processor 2120. The computing device 2100, or components thereof, can be used to perform any of the systems and methods detailed herein. The computing device 2100 can include a cache of high-speed memory connected directly with, in close proximity to, or integrated as part of the processor 2120. The computing device 2100 copies data from the memory 2130 and/or the storage device 2160 to the cache for quick access by the processor 2120. In this way, the cache provides a performance boost that avoids processor 2120 delays while waiting for data. These and other modules can control or be configured to control the processor 2120 to perform various actions. Other memory 2130 may be available for use as well. The memory 2130 can include multiple different types of memory with different performance characteristics. It can be appreciated that the disclosure may operate on a computing device 2100 with more than one processor 2120 or on a group or cluster of computing devices networked together to provide greater processing capability. The processor 2120 can include any general-purpose processor and a hardware module or software module, such as module 1 2162, module 2 2164, and module 3 2166 stored in storage device 2160, configured to control the processor 2120 as well as a special-purpose processor where software instructions are incorporated into the actual processor design. The processor 2120 may essentially be a completely self-contained computing system, containing multiple cores or processors, a bus, memory controller, cache, etc. A multi-core processor may be symmetric or asymmetric.

The system bus 2110 may be any of several types of bus structures including a memory bus or memory controller, a peripheral bus, and a local bus using any of a variety of bus architectures. A basic input/output (BIOS) stored in ROM 2140 or the like, may provide the basic routine that helps to transfer information between elements within the computing device 2100, such as during start-up. The computing device 2100 further includes storage devices 2160 such as a hard disk drive, a magnetic disk drive, an optical disk drive, tape drive or the like. The storage device 2160 can include software modules 2162, 2164, 2166 for controlling the processor 2120. Other hardware or software modules are contemplated. The storage device 2160 is connected to the system bus 2110 by a drive interface. The drives and the associated computer-readable storage media provide nonvolatile storage of computer-readable instructions, data structures, program modules and other data for the computing device 2100. In one aspect, a hardware module that performs a particular function includes the software component stored in a tangible computer-readable storage medium in connection with the necessary hardware components, such as the processor 2120, system bus 2110, output device 2170, and so forth, to carry out the function. In another aspect, the system can use a processor and computer-readable storage medium to store instructions which, when executed by a processor (e.g., one or more processors), cause the processor to perform a method or other specific actions. The basic components and appropriate variations are contemplated depending on the type of device, such as whether the computing device 2100 is a small, handheld computing device, a desktop computer, or a computer server.

Although the exemplary embodiment described herein employs the storage device 2160, other types of computer-readable media which can store data that are accessible by a computer, such as magnetic cassettes, flash memory cards, digital versatile disks, cartridges, random-access memories (RAMs) 2150, and read-only memory (ROM) 2140, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 2100, an input device 2190 represents any number of input mechanisms, such as a microphone for speech, a touch-sensitive screen for gesture or graphical input, keyboard, mouse, motion input, speech and so forth. An output device 2170 can also be one or more of a number of output mechanisms known to those of skill in the art, such as, for example, a display. In some instances, multimodal systems enable a user to provide multiple types of input to communicate with the computing device 2100. The communications interface 2180 generally governs and manages the user input and system output. There is no restriction on operating on any particular hardware arrangement and therefore the basic features here may easily be substituted for improved hardware or firmware arrangements as they are developed.

The technology discussed herein refers to computer-based systems and actions taken by, and information sent to and from, computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel

Further aspects are provided by the subject matter of the following clauses.

A system for monitoring one or more addictive activities by an addict, the system including at least one biosensor configured to measure physiological data from the addict, a processor configured to generate a notification when the addict has participated in the one or more addictive activities based on the physiological data, and a communication system configured to send the notification to one or more devices of the addict and/or of members of an addiction support network of the addict to notify that the addict has participated in the addictive activities.

The system of the preceding clause, wherein the at least one biosensor is attached to the addict.

The system of any preceding clause, wherein the at least one biosensor is combined into a housing and attached to the addict.

The system of any preceding clause, wherein the at least one biosensor is contained in a band-mounted wearable device that is worn by the addict.

The system of any preceding clause, wherein the at least one biosensor is contained in an adhesive-based wearable device that is attached to the addict.

The system of any preceding clause, wherein the at least one biosensor is configured to transmit biosensor data of the addict to the processor via wireless communication or wired communication.

The system of any preceding clause, wherein the processor is configured to process the biosensor data received from the at least one biosensor to identify and characterize artifacts, to extract candidate features for classification and storage and/or to compare to previously acquired candidate features, and to generate a report.

The system of any preceding clause, wherein the processor is configured to determine if the addict has participated in one or more addictive activities.

The system of any preceding clause, wherein the processor is configured to determine if the addict has signs of withdrawal or addictions.

The system of any preceding clause, wherein the processor is configured to determine if the addict has urges for the one or more addictive activities.

The system of any preceding clause, wherein the processor is configured to determine if the addict is compliant with a prescription.

The system of any preceding clause, wherein the processor is configured to determine if the addict is exhibiting signs of detoxification or withdrawal of the one or more addictive activities.

The system of any preceding clause, wherein the processor is configured to determine if the addict is experiencing adverse events from participating in the one or more addictive activities.

The system of any preceding clause, wherein the processor is configured to determine if the addict is engaged in a secondary support associated activity.

The system of any preceding clause, wherein the processor is configured to determine whether to send the biosensor data to members of the addiction support network of the addict, and send the biosensor data to the one or more devices of the members of the addiction support network upon determining to send the biosensor data to the members of the addiction support network.

The system of any preceding clause, further comprising a wearable device containing the at least one biosensor, the wearable device being worn by the addict, wherein the processor is configured to display the notification on the wearable device and/or transmit the notification to members of the addiction support network.

The system of any preceding clause, wherein the communication system is configured to communicate with an artificial intelligence-driven human-like avatar or bot.

The system of any preceding clause, wherein the processor is configured to automatically determine when a parameter of the addict from the at least one biosensor is out of range due to participation in addictive activities, and automatically establish, via the communication system, uni-directional or bi-directional communication with the addict to send the notification.

The system of any preceding clause, wherein the processor is configured to initiate auto-injection of antidote substances to counter consumption of addictive substances by the addict.

The system of any preceding clause, wherein the processor is configured to monitor physiological parameters in the physiological data in a time series analysis using at least one of logistic regression/classification, discriminant analysis, tree-based methods, fuzzy logic, genetic algorithms, or machine learning.

The system of any preceding clause, wherein the processor is configured to disable heavy machinery, cars, planes, trains, boats, or dangerous equipment around the addict if the addict has participated in one or more addictive activities.

The system of any preceding clause, wherein the processor is configured to determine whether the addict is unfit for duty if the addict has participated in one or more addictive activities, and notify, via the communication system, members of the addiction support network that the addict is unfit for duty.

The system of any preceding clause, wherein the at least one biosensor is configured to record the physiological data.

The system of any preceding clause, wherein the processor is configured to analyze the physiological data and determine when the addict has participated in the one or more addictive activities.

The system of any preceding clause, wherein the processor is configured to analyze the physiological data in real-time.

The system of any preceding clause, wherein the notification is an alarm or an alert.

The system of any preceding clause, wherein the notification enables the addict or the member of the addiction support network to intervene and/or prevent the addict from participating in the one or more addictive activities.

The system of any preceding clause, wherein the processor is a virtual sobriety partner for the addict, and the processor is configured to communicate with either a human or an artificial intelligence-based engine.

The system of any preceding clause, wherein the processor is configured to intervene with the addict to prevent or minimize participation in the one or more addictive activities.

The system of any preceding clause, further including at least one geographical location sensor configured to measure geographical location or proximity data of the addict, and the processor is configured to notify, via the communication system, the addict that the addict has entered a defined dangerous geographical zone or exited a defined safe geographical zone based on the geographical location or proximity data.

The system of any preceding clause, further comprising a mobile system containing the at least on biosensor, wherein the processor is configured to notify, via the communication system, the addict and/or an addiction support network of the addict that the mobile system is not being worn by the addict based on the physiological data.

A system for providing a virtual sobriety partner for an addict, the system including at least one biosensor configured to measure physiological data from the addict; and a processor configured to communicate with one or more devices of either a human or an artificial intelligence-based engine to notify the human or the artificial intelligence-based engine that the addict has participated in one or more addictive activities based on the physiological data.

The system of the preceding clause, wherein the processor is configured to conduct advanced therapy with the addict.

The system of any preceding clause, wherein the advanced therapy includes at least one of cognitive-behavioral therapy, talk therapy, self-management and recovery training (SMART) recovery or cost-benefit therapy, or 12-step therapy.

The system of any preceding clause, wherein the processor is configured to analyze the physiological data, and determine when the addict has participated in one or more addictive activities based on the physiological data.

The system of any preceding clause, wherein the processor is configured to analyze the physiological data in real-time.

The system of any preceding clause, wherein the processor is configured to intervene with the addict to prevent or minimize participation in the addictive activities.

The system of any preceding clause, wherein the at least one biosensor is configured to record the physiological data.

A system for monitoring a risk of participation in one or more addictive activities by an addict using geographical location or proximity data, the system including at least one biosensor configured to measure physiological data from the addict, at least one geographical location sensor configured to measure geographical location or proximity data of the addict, a processor configured to generate a notification when the addict has either entered a defined dangerous geographical zone or exited a defined safe geographical zone, and a communication system configured to send the notification to one or more devices of the addict to notify that the addict has entered the defined dangerous geographical zone or exited the defined safe geographical zone.

The system of the preceding clause, wherein the processor is configured to send a notification to one or more devices of members of an addiction support network of the addict that the addict has entered the defined dangerous geographical zone or exited the defined safe geographical zone to enable the members of the addiction support network to intervene and minimize the addictive activities by the addict.

The system of any preceding clause, wherein the processor is configured to combine the geographical location or proximity data with the physiological data, and notify first responders to intervene and improve health of the addict.

The system of any preceding clause, wherein the geographical location sensor is a Global Positioning System sensor (GPS), a Bluetooth transceiver, a Wi-Fi transceiver, or an ultra-wide band location sensor.

The system of any preceding clause, wherein the defined dangerous geographical zone and the defined safe geographical zone are user-defined in both space and time.

The system of any preceding clause, wherein the defined dangerous geographical zone and the defined safe geographical zone change based on a time of day and/or a spatial proximity for both fixed and moving risks for addictive activities of the addict.

The system of any preceding clause, wherein the processor is configured to determine if the addict is in a place conducive to participating in addictive activities.

The system of any preceding clause, wherein the processor is configured to transmit geographical location information to one or more devices of members of an addiction support network of the addict to enable the members of the addiction support network to intervene and prevent the addict from participating in the addictive activities.

The system of any preceding clause, wherein the processor is configured to analyze the geographical location or proximity data, and determine when the addict has either entered the defined dangerous geographical zone or exited the defined safe geographical zone based on the geographical location or proximity data.

The system of any preceding clause, wherein the processor is configured to analyze the geographical location or proximity data in real-time.

A mobile system for identifying an addict that uses the mobile system to prevent an unauthorized use of the mobile system by another other than the addict, the mobile system including at least one biosensor configured to measure physiological data from the addict, and a processor configured to send, via a communication system, a notification to one or more devices of the addict and/or of members of an addiction support network of the addict that the mobile system is not being worn by the addict based on the physiological data.

The mobile system of the preceding clause, wherein the processor is configured to compare the physiological data to baseline measurements, and determine if the mobile system is not being worn by the addict based on the comparison of the physiological data to the baseline measurements.

The mobile system of any preceding clause, wherein the processor is configured to analyze the physiological data, and determine when the mobile system is not being worn by the addict based on the physiological data.

The mobile system of any preceding clause, wherein the processor is configured to analyze the physiological data in real-time.

The mobile system of any preceding clause, wherein the processor is configured to communicate with the one or more devices via uni-directional or bi-directional communication to enable intervention and/or restoration of the mobile system on the addict

A method for monitoring one or more addictive activities by an addict, the method including measuring, with at least one biosensor, physiological data from the addict, generating, by a processor, a notification when the addict has participated in one or more addictive activities based on the physiological data, and sending, by the processor, the notification to one or more devices of the addict and/or of members of an addiction support network of the addict to notify that the addict has participated in one or more addictive activities.

The method of the preceding clause, wherein the at least one biosensor is attached to the addict.

The method of any preceding clause, wherein the at least one biosensor is combined into a housing and attached to the addict.

The method of any preceding clause, wherein the at least one biosensor is contained in a band-mounted wearable device that is worn by the addict.

The method of any preceding clause, wherein the at least one biosensor is contained in an adhesive-based wearable device that is attached to the addict.

The method of any preceding clause, further comprising transmitting, by the at least one biosensor, biosensor data of the addict to the processor via wireless communication or wired communication.

The method of any preceding clause, further comprising processing, by the processor, the biosensor data received from the at least one biosensor to identify and characterize artifacts, to extract candidate features for classification and storage and/or to compare to previously acquired candidate features, and to generate a report.

The method of any preceding clause, further comprising determining, by the processor, if the addict has participated in addictive activities.

The method of any preceding clause, further comprising determining, by the processor, if the addict has signs of withdrawal or addictions.

The method of any preceding clause, further comprising determining, by the processor, if the addict has urges for the addictive activities.

The method of any preceding clause, further comprising determining, by the processor, if the addict is compliant with a prescription.

The method of any preceding clause, further comprising determining, by the processor, if the addict is exhibiting signs of detoxification or withdrawal of the one or more addictive activities.

The method of any preceding clause, further comprising determining, by the processor, if the addict is experiencing adverse events from participating in the one or more addictive activities.

The method of any preceding clause, further comprising determining, by the processor, if the addict is engaged in a secondary support associated activity.

The method of any preceding clause, further comprising determining, by the processor, whether to send the biosensor data to members of the addiction support network of the addict, and sending, by the server, the biosensor data to the one or more devices of the members of the addiction support network upon determining to send the biosensor data to the members of the addiction support network.

The method of any preceding clause, further comprising displaying, by the processor, the notification on a wearable device containing the at least one biosensor and being worn by the addict, and/or transmitting, by the server, the notification to members of the addiction support network.

The method of any preceding clause, further comprising communicating, by the processor, with an artificial intelligence-driven human-like avatar or bot.

The method of any preceding clause, further comprising automatically determining, by the processor, when a parameter of the addict from the at least one biosensor is out of range due to participation in addictive activities, and automatically establishing, by the processor via uni-directional or bi-directional communication with the addict, to send the notification.

The method of any preceding clause, further comprising initiating, by the processor, auto-injection of antidote substances to counter consumption of addictive substances by the addict.

The method of any preceding clause, further comprising monitoring, by the processor, physiological parameters in a time series analysis using at least one of logistic regression/classification, discriminant analysis, tree-based methods, fuzzy logic, genetic algorithms, or machine learning.

The method of any preceding clause, further comprising disabling, by the processor, heavy machinery, cars, planes, trains, boats, or dangerous equipment around the addict if the addict has participated in one or more addictive activities.

The method of any preceding clause, further comprising determining, by the processor, whether the addict is unfit for duty if the addict has participated in addictive activities, and notifying, by the processor, members of the addiction support network that the addict is unfit for duty.

The method of any preceding clause, wherein the at least one biosensor records the physiological data.

The method of any preceding clause, wherein the processor analyzes the physiological data and determines when the addict has participated in the one or more addictive activities.

The method of any preceding clause, wherein the processor analyzes the physiological data in real-time.

The method of any preceding clause, wherein the notification is an alarm or an alert.

The method of any preceding clause, wherein the notification enables the addict or the member of the addiction support network to intervene and/or prevent the addict from participating in the one or more addictive activities.

The method of any preceding clause, wherein the processor is a virtual sobriety partner for the addict, and communicates with either a human or an artificial intelligence-based engine.

The method of any preceding clause, wherein the processor intervenes with the addict to prevent or minimize participation in the one or more addictive activities.

The method of any preceding clause, further including at least one geographical location sensor that measures geographical location or proximity data of the addict, and the processor notifies, via the communication system, the addict that the addict has entered a defined dangerous geographical zone or exited a defined safe geographical zone based on the geographical location or proximity data.

The method of any preceding clause, wherein the processor notifies, via the communication system, the addict and/or an addiction support network of the addict that the mobile system is not being worn by the addict based on the physiological data.

A method for providing a virtual sobriety partner for an addict, the method including measuring, by at least one biosensor, physiological data from the addict, and communicating, by the processor, with one or more devices of either a human or an artificial intelligence-based engine to notify the human or the artificial intelligence-based engine that the addict has participated in one or more addictive activities based on the physiological data.

The method of any preceding clause, further comprising conducting, by the processor, advanced therapy with the addict.

The method of any preceding clause, wherein the advanced therapy includes at least one of cognitive-behavioral therapy, talk therapy, self-management and recovery training (SMART) recovery or cost-benefit therapy, or 12-step therapy.

The method of any preceding clause, wherein the processor analyzes the physiological data, and determines when the addict has participated in one or more addictive activities based on the physiological data.

The method of any preceding clause, wherein the processor analyzes the physiological data in real-time.

The method of any preceding clause, wherein the processor intervenes with the addict to prevent or minimize participation in the addictive activities.

The method of any preceding clause, wherein the at least one biosensor records the physiological data.

A method for monitoring a risk of participation in one or more addictive activities by an addict using geographical location or proximity data, the method including measuring, by at least one biosensor, physiological data from the addict, measuring, by at least one geographical location sensor, geographical location or proximity data of the addict, generating, by a processor, a notification when the addict has either entered a defined dangerous geographical zone or exited a defined safe geographical zone, and sending, by the processor via a communication system, the notification to one or more devices of the addict to notify that the addict has entered the defined dangerous geographical zone or exited the defined safe geographical zone.

The method of any preceding clause, further comprising sending, by the processor, a notification to one or more devices of members of an addiction support network of the addict that the addict has entered the defined dangerous geographical zone or exited the defined safe geographical zone to enable the members of the addiction support network to intervene and minimize the addictive activities by the addict.

The method of any preceding clause, further comprising combining, by the processor, the geographical location or proximity data with the physiological data, and notifying, by the processor, first responders to intervene and improve health of the addict.

The method of any preceding clause, wherein the geographical location sensor is a Global Positioning System sensor (GPS), a Bluetooth transceiver, a Wi-Fi transceiver, or an ultra-wide band location sensor.

The method of any preceding clause, wherein the defined dangerous geographical zone and the defined safe geographical zone are user-defined in both space and time.

The method of any preceding clause, wherein the defined dangerous geographical zone and the defined safe geographical zone change based on a time of day and/or a spatial proximity for both fixed and moving risks for addictive activities of the addict.

The method of any preceding clause, further comprising determining, by the processor, if the addict is in a place conducive to participating in addictive activities.

The method of any preceding clause, further comprising transmitting, by the processor, geographical location information to members of an addiction support network of the addict to enable the members of the addiction support network to intervene and prevent the addict from participating in the addictive activities.

The method of any preceding clause, wherein the processor analyzes the geographical location or proximity data, and determines when the addict has either entered the defined dangerous geographical zone or exited the defined safe geographical zone based on the geographical location or proximity data.

The method of any preceding clause, wherein the processor analyzes the geographical location or proximity data in real-time.

A method for identifying an addict that uses a mobile system to prevent an unauthorized use of the mobile system by another other than the addict, the method including measuring, by at least one biosensor, physiological data from the addict, sending, by a processor via a communication system, a notification to one or more devices of the addict and/or of members of an addiction support network of the addict that the mobile system is not being worn by the addict based on the physiological data.

The method of any preceding clause, further comprising comparing, by the processor, the physiological data to baseline measurements, and determining, by the processor, if the mobile system is not being worn by the addict based on the comparison of the physiological data to the baseline measurements.

The method of any preceding clause, wherein the processor analyzes the physiological data, and determines when the mobile system is not being worn by the addict based on the physiological data.

The method of any preceding clause, wherein the processor analyzes the physiological data in real-time.

The method of any preceding clause, wherein the processor communicates with the one or more devices via uni-directional or bi-directional communication to enable intervention and/or restoration of the mobile system on the addict.

A method for monitoring one or more addictive activities by an addict, the method including receiving, by a processor, physiological data of an addict from at least one biosensor, and sending, by the processor, a notification to one or more devices of an addict when the addict has participated in one or more addictive activities based on the physiological data.

The method of the preceding clause, further comprising determining, by the processor, that the addict has participated in the one or more addictive activities based on the physiological data.

The method of any preceding clause, further comprising generating, by the processor, the notification when the addict has participated in the one or more addictive activities.

The method of the preceding clause, wherein the at least one biosensor is attached to the addict.

The method of any preceding clause, wherein the at least one biosensor is combined into a housing and attached to the addict.

The method of any preceding clause, wherein the at least one biosensor is contained in a band-mounted wearable device that is worn by the addict.

The method of any preceding clause, wherein the at least one biosensor is contained in an adhesive-based wearable device that is attached to the addict.

The method of any preceding clause, further comprising receiving, by the processor, the physiological data of the addict from the at least one biosensor via wireless communication or wired communication.

The method of any preceding clause, further comprising processing, by the processor, the biosensor data received from the at least one biosensor to identify and characterize artifacts, to extract candidate features for classification and storage and/or to compare to previously acquired candidate features, and to generate a report.

The method of any preceding clause, further comprising determining, by the processor, if the addict has participated in addictive activities.

The method of any preceding clause, further comprising determining, by the processor, if the addict has signs of withdrawal or addictions.

The method of any preceding clause, further comprising determining, by the processor, if the addict has urges for the addictive activities.

The method of any preceding clause, further comprising determining, by the processor, if the addict is compliant with a prescription.

The method of any preceding clause, further comprising determining, by the processor, if the addict is exhibiting signs of detoxification or withdrawal of the one or more addictive activities.

The method of any preceding clause, further comprising determining, by the processor, if the addict is experiencing adverse events from participating in the one or more addictive activities.

The method of any preceding clause, further comprising determining, by the processor, if the addict is engaged in a secondary support associated activity.

The method of any preceding clause, further comprising determining, by the processor, whether to send the biosensor data to members of the addiction support network of the addict, and sending, by the server, the biosensor data to the one or more devices of the members of the addiction support network upon determining to send the biosensor data to the members of the addiction support network.

The method of any preceding clause, further comprising causing, by the processor, a wearable device containing the at least one biosensor to display the notification on the wearable device, the wearable device being worn by the addict.

The method of any preceding clause, further comprising transmitting, by the processor, the notification to members of the addiction support network.

The method of any preceding clause, further comprising communicating, by the processor, with an artificial intelligence-driven human-like avatar or bot.

The method of any preceding clause, further comprising automatically determining, by the processor, when a parameter of the addict from the at least one biosensor is out of range due to participation in addictive activities, and automatically establishing, by the processor via uni-directional or bi-directional communication with the addict, to send the notification.

The method of any preceding clause, further comprising initiating, by the processor, auto-injection of antidote substances to counter consumption of addictive substances by the addict.

The method of any preceding clause, further comprising monitoring, by the processor, physiological parameters in a time series analysis using at least one of logistic regression/classification, discriminant analysis, tree-based methods, fuzzy logic, genetic algorithms, or machine learning.

The method of any preceding clause, further comprising disabling, by the processor, heavy machinery, cars, planes, trains, boats, or dangerous equipment around the addict if the addict has participated in one or more addictive activities.

The method of any preceding clause, further comprising determining, by the processor, whether the addict is unfit for duty if the addict has participated in addictive activities, and notifying, by the processor, members of the addiction support network that the addict is unfit for duty.

The method of any preceding clause, wherein the at least one biosensor records the physiological data.

The method of any preceding clause, wherein the processor analyzes the physiological data and determines when the addict has participated in the one or more addictive activities.

The method of any preceding clause, wherein the processor analyzes the physiological data in real-time.

The method of any preceding clause, wherein the notification is an alarm or an alert.

The method of any preceding clause, wherein the notification enables the addict or the member of the addiction support network to intervene and/or prevent the addict from participating in the one or more addictive activities.

The method of any preceding clause, wherein the processor is a virtual sobriety partner for the addict, and communicates with either a human or an artificial intelligence-based engine.

The method of any preceding clause, wherein the processor intervenes with the addict to prevent or minimize participation in the one or more addictive activities.

The method of any preceding clause, further including at least one geographical location sensor that measures geographical location or proximity data of the addict, and the processor notifies, via the communication system, the addict that the addict has entered a defined dangerous geographical zone or exited a defined safe geographical zone based on the geographical location or proximity data.

The method of any preceding clause, wherein the processor notifies, via the communication system, the addict and/or an addiction support network of the addict that the mobile system is not being worn by the addict based on the physiological data.

A tangible non-transitory computer-readable storage medium having instructions stored which, when executed by at least one processor, cause the at least one processor to perform operations comprising the method of any preceding clause.

A system for monitoring the consumption of addictive substances or participation in addictive behaviors by a subject, comprising at least one biosensor that measures and records physiological data from the subject, a central processing unit that analyzes the physiological data in real-time and determines when the subject has consumed addictive substances or participated in addictive behaviors, and a communication system with the means to notify the subject and/or their addiction support network of the detrimental activity, and provides means for either uni-directional (1-way) or bi-directional (2-way) communication from the subject which intervenes and improves the care of the subject.

The system of the preceding clause, further comprising a biosensor attached to a subject.

The system of any preceding clause, further comprising a biosensor combined into one or more housing and attached to a subject.

The system of any preceding clause, further comprising a biosensor in a housing worn on the subject's wrist.

The system of any preceding clause, further comprising a biosensor attached adhesively or mechanically to the subject's body.

The system of any preceding clause, further comprising a biosensor that transmits sensor signals to a server via wireless or wired means.

The system of any preceding clause, wherein said server processes biological sensor data received from said electronic module to identify and characterize artifacts, to extract candidate features for classification and storage and/or for comparison to previously acquired candidate features, and to generate a report.

The system of any preceding clause, wherein the server algorithm determines if the subject has engaged in addictive behavior or activities.

The system of any preceding clause, wherein the server determines if the subject has signs of withdrawal or addictions.

The system of any preceding clause, wherein the server determines if the subject has urges for the addictive behavior.

The system of any preceding clause, wherein the server determines if the subject is compliant with a prescription.

The system of any preceding clause, wherein the server determines if a subject is exhibiting signs of detoxification or withdrawal of the addictive behavior or substance.

The system of any preceding clause, wherein the server determines if a subject is experiencing adverse events from the consumption of addictive substances or engaging in addictive behaviors.

The system of any preceding clause, wherein the server determines if a subject is engaged in a secondary support associated activity.

The system of any preceding clause, wherein the server determines if biosensor data should be sent to members of the subject's addiction support network.

The system of any preceding clause, wherein the server determines if alarms should be displayed on the wearer's device or transmitted to members of the addiction support network.

The system of any preceding clause, further comprising a communication system that can have single way communication or bi-directional communication with the subject, or communication with an artificial intelligence-driven human-like avatar or bot.

The system of any preceding clause, further comprising an automated analysis that can determine when a parameter is out of range due to consumption of an addictive substance or participation in addictive behavior and can automatically trigger the communication system to have uni-directional (1-way) or bi-directional (2-way) communication with the subject.

The system of any preceding clause, further comprising the means to initiate the auto-injection of Narcan or other antidote substances to counter the consumption of addictive substances.

The system of any preceding clause, further comprising analysis that monitors the physiological parameters with time series analysis amongst logistic regression/classification, discriminant analysis, tree-based methods, fuzzy logic, genetic algorithms, machine learning or any other predictive statistical method or model.

The system of any preceding clause, further comprising an intervention whereby if the subject has consumed addictive substances or engaged in addictive behaviors, then the system can disable heavy machinery, cars, planes, trains, boats, or dangerous equipment around them.

The system of any preceding clause, further comprising an intervention whereby if the subject has consumed addictive substances or engaged in addictive behaviors, then the system can determine that the subject is not fit for duty and notification of the addictive support network.

A system that acts as a virtual sobriety partner, comprising at least one biosensor that measures and records physiological data from the subject, a central processing unit that analyzes the physiological data in real-time and determines when the subject has consumed addictive substances or participated in addictive behaviors, and the means to notify the subject of the potential consumption or participation, and the means to engage in 2-way communication with either a human or an artificial intelligence-based recovery engine serving as a virtual avatar for the subject, and the means to intervene with the subject to prevent or minimize substance consumption or behavior participation, and optionally conducts advanced therapy with the subject.

The system of the preceding clause, further comprising advanced therapy in the form of cognitive-behavioral therapy, talk therapy, SMART recovery or cost-benefit therapy, and 12-step therapy.

A system for monitoring and detecting the risk of consumption of addictive substances or participation in addictive behaviors by a subject using geographical location or proximity data, comprising at least one biosensor that measures and records physiological data from the subject, and at least one geographical location sensor that measures and records geographical location or proximity data from the subject, and a central processing unit that analyzes the geographical location data in real-time and determines when the subject has either entered a defined dangerous geographical zone or exited a defined safe geographical zone, and the means to notify the subject of the geographical breach, and alternatively has the means to notify the subject's addiction support network of the geographical breach to enable intervention and the minimization of detrimental substance consumption or behavior participation.

The system of the preceding clause, wherein said analysis combines geographical location or proximity data with physiological adverse event data to notify first responders to intervene and improve health.

The system of any preceding clause, wherein the geographical location sensor is a Global Positioning System sensor (GPS), a Bluetooth transceiver, a Wi-Fi transceiver or an ultra-wide band location sensor or equivalent.

The system of any preceding clause, wherein the defined dangerous or safe geographical zones are user defined in both space and time, as they are based on the individual subject's needs, and can change depending on the time of day as well as spatial proximity for both fixed and moving risks, such as a spouse or a drug dealer or a highway rest area.

The system of any preceding clause, wherein the analysis determines if the subject is in a place conducive to consuming addictive substances or engaging in or supporting addictive behaviors.

The system of any preceding clause, wherein the system transmits geographical location information to the addiction support network which enables intervention.

A mobile system that can uniquely identify the subject of the system to prevent the unauthorized use of the system by another other than the subject, comprising at least one biosensor that measures and records physiological data from the subject, and a central processing unit that analyzes the physiological data in real-time and determines when the system is not being worn by the subject, and the means to notify the subject and/or their addiction support network that the system is no longer being worn by the subject, and provides means for either 1-way or 2-way communication from the subject which enables intervention and restoration of the system on the subject.

The system of any preceding clause, wherein a comparison to baseline measurements is conducted which can determine if the system is not being worn by anyone.

A method for monitoring the consumption of addictive substances or participation in addictive behaviors by a subject, comprising measuring and recording, by at least one biosensor, physiological data from the subject, analyzing, by a central processing unit, the physiological data in real-time, determining when the subject has consumed addictive substances or participated in addictive behaviors, and notifying, by a communication system, the subject and/or their addiction support network of the detrimental activity by either uni-directional (1-way) or bi-directional (2-way) communication from the subject which intervenes and improves the care of the subject.

A method for providing a virtual sobriety partner, comprising measuring and recording, by at least one biosensor, physiological data from the subject, analyzing, by a central processing unit, the physiological data in real-time, determining when the subject has consumed addictive substances or participated in addictive behaviors, notifying the subject of the potential consumption or participation, engaging in 2-way communication with either a human or an artificial intelligence-based recovery engine serving as a virtual avatar for the subject, and intervening with the subject to prevent or minimize substance consumption or behavior participation, and optionally conducting advanced therapy with the subject.

A method for monitoring and detecting the risk of consumption of addictive substances or participation in addictive behaviors by a subject using geographical location or proximity data, comprising measuring and recording, by at least one biosensor, physiological data from the subject, measuring and recording, by at least one geographical location sensor, geographical location or proximity data from the subject, analyzing, by a central processing unit, the geographical location data in real-time, determining, by the central processing unit, when the subject has either entered a defined dangerous geographical zone or exited a defined safe geographical zone, and notifying the subject of the geographical breach, and alternatively notifying the subject's addiction support network of the geographical breach to enable intervention and the minimization of detrimental substance consumption or behavior participation.

A method for uniquely identifying the subject of the system to prevent the unauthorized use of the system by another other than the subject, comprising measuring and recording, by at least one biosensor, physiological data from the subject, analyzing, by a central processing unit, the physiological data in real-time, determining, by the central processing unit, when the system is not being worn by the subject, and notifying, by the central processing unit, the subject and/or their addiction support network that the system is no longer being worn by the subject by either 1-way or 2-way communication from the subject which enables intervention and restoration of the system on the subject.

The method of any preceding clause, further comprising the system of any preceding clause.

A tangible non-transitory computer-readable storage medium having instructions stored which, when executed by at least one processor, cause the at least one processor to perform operations comprising the method of any preceding clause.

Although the foregoing description is directed to the preferred embodiments of the present disclosure, other variations and modifications will be apparent to those skilled in the art and may be made without departing from the spirit or the scope of the disclosure. For example, the signal processing described herein may be performed on a server, in the cloud, in the electronics module, or on a local PC, tablet PC, smartphone, or custom handheld device. Accordingly, the scope of the present disclosure is not intended to be limited to the exemplary embodiments described above, but only by the appended claims. Moreover, features described in connection with one embodiment of the present disclosure may be used in conjunction with other embodiments, even if not explicitly stated above.

1. A system for monitoring one or more addictive activities by an addict, the system comprising:
at least one biosensor configured to measure physiological data from the addict;
a processor configured to generate a notification when the addict has participated in the one or more addictive activities based on the physiological data; and
a communication system configured to send the notification to one or more devices of the addict and/or of members of an addiction support network of the addict to notify that the addict has participated in the addictive activities.

2. The system of claim 1, wherein the at least one biosensor is attached to the addict.

3. The system of claim 1, wherein the at least one biosensor is combined into a housing and attached to the addict.

4. The system of claim 1, wherein the at least one biosensor is contained in a band-mounted wearable device that is worn by the addict.

5. The system of claim 1, wherein the at least one biosensor is contained in an adhesive-based wearable device that is attached to the addict.

6. The system of claim 1, wherein the at least one biosensor is configured to transmit biosensor data of the addict to the processor via wireless communication or wired communication.

7. The system of claim 6, wherein the processor is configured to process the biosensor data received from the at least one biosensor to identify and characterize artifacts, to extract candidate features for classification and storage and/or to compare to previously acquired candidate features, and to generate a report.

8. The system of claim 6, wherein the processor is configured to determine if the addict has participated in one or more addictive activities.

9. The system of claim 6, wherein the processor is configured to determine if the addict has signs of withdrawal or addictions.

10. The system of claim 6, wherein the processor is configured to determine if the addict has urges for the one or more addictive activities.

11. The system of claim 6, wherein the processor is configured to determine if the addict is compliant with a prescription.

12. The system of claim 6, wherein the processor is configured to determine if the addict is exhibiting signs of detoxification or withdrawal of the one or more addictive activities.

13. The system of claim 6, wherein the processor is configured to determine if the addict is experiencing adverse events from participating in the one or more addictive activities.

14. The system of claim 6, wherein the processor is configured to determine if the addict is engaged in a secondary support associated activity.

15. The system of claim 6, wherein the processor is configured to determine whether to send the biosensor data to members of the addiction support network of the addict, and sends the biosensor data to the one or more devices of the members of the addiction support network upon determining to send the biosensor data to the members of the addiction support network.

16. The system of claim 6, further comprising a wearable device containing the at least one biosensor, the wearable device being worn by the addict, wherein the processor is configured to display the notification on the wearable device and/or transmit the notification to members of the addiction support network.

17. The system of claim 1, wherein the communication system is configured to communicate with an artificial intelligence-driven human-like avatar or bot.

18. The system of claim 1, wherein the processor is configured to automatically determine when a parameter of the addict from the at least one biosensor is out of range due to participation in addictive activities, and automatically establish, via the communication system, uni-directional or bi-directional communication with the addict to send the notification.

19. The system of claim 1, wherein the processor is configured to initiate auto-injection of antidote substances to counter consumption of addictive substances by the addict.

20. The system of claim 1, wherein the processor is configured to monitor physiological parameters in the physiological data in a time series analysis using at least one of logistic regression/classification, discriminant analysis, tree-based methods, fuzzy logic, genetic algorithms, or machine learning.

21. The system of claim 1, wherein the processor is configured to disable heavy machinery, cars, planes, trains, boats, or dangerous equipment around the addict if the addict has participated in one or more addictive activities.

22. The system of claim 1, wherein the processor is configured to determine whether the addict is unfit for duty if the addict has participated in one or more addictive activities, and notify, via the communication system, members of the addiction support network that the addict is unfit for duty.

23. The system of claim 1, wherein the at least one biosensor is configured to record the physiological data.

24. The system of claim 1, wherein the processor is configured to analyze the physiological data and determine when the addict has participated in the one or more addictive activities.

25. The system of claim 24, wherein the processor is configured to analyze the physiological data in real-time.

26. The system of claim 1, wherein the notification is an alarm or an alert.

27. The system of claim 1, wherein the notification enables the addict or the members of the addiction support network to intervene and/or prevent the addict from participating in the one or more addictive activities.

28. The system of claim 1, wherein the processor is a virtual sobriety partner for the addict, and the processor is configured to communicate with either a human or an artificial intelligence-based engine.

29. The system of claim 1, wherein the processor is configured to intervene with the addict to prevent or minimize participation in the one or more addictive activities.

30. The system of claim 1, further including at least one geographical location sensor that is configured to measure geographical location or proximity data of the addict, and the processor is configured to notify, via the communication system, the addict that the addict has entered a defined dangerous geographical zone or exited a defined safe geographical zone based on the geographical location or proximity data.

31. The system of claim 1, further comprising a mobile system containing the at least on biosensor, wherein the processor is configured to notify, via the communication system, the addict and/or an addiction support network of the addict that the mobile system is not being worn by the addict based on the physiological data.

32. A system for providing a virtual sobriety partner for an addict, the system comprising:
at least one biosensor configured to measure physiological data from the addict; and
a processor configured to communicate with one or more devices of either a human or an artificial intelligence-based engine to notify the human or the artificial intelligence-based engine that the addict has participated in one or more addictive activities based on the physiological data.

33. The system of claim 32, wherein the processor is configured to conduct advanced therapy with the addict.

34. The system of claim 33, wherein the advanced therapy includes at least one of cognitive-behavioral therapy, talk therapy, self-management and recovery training (SMART) recovery or cost-benefit therapy, or 12-step therapy.

35. The system of claim 32, wherein the processor is configured to analyze the physiological data, and determine when the addict has participated in one or more addictive activities based on the physiological data.

36. The system of claim 35, wherein the processor is configured to analyze the physiological data in real-time.

37. The system of claim 32, wherein the processor is configured to intervene with the addict to prevent or minimize participation in the addictive activities.

38. The system of claim 32, wherein the at least one biosensor is configured to the physiological data.

39. A system for monitoring a risk of participation in one or more addictive activities by an addict using geographical location or proximity data, the system comprising:
at least one biosensor configured to measure physiological data from the addict;
at least one geographical location sensor configured to measure geographical location or proximity data of the addict;
a processor configured to generate a notification when the addict has either entered a defined dangerous geographical zone or exited a defined safe geographical zone; and
a communication system configured to send the notification to one or more devices of the addict to notify that the addict has entered the defined dangerous geographical zone or exited the defined safe geographical zone.

40. The system of claim 39, wherein the processor is configured to send a notification to one or more devices of members of an addiction support network of the addict that the addict has entered the defined dangerous geographical zone or exited the defined safe geographical zone to enable the members of the addiction support network to intervene and minimize the addictive activities by the addict.

41. The system of claim 39, wherein the processor is configured to combine the geographical location or proximity data with the physiological data, and notify first responders to intervene and improve health of the addict.

42. The system of claim 39, wherein the geographical location sensor is a Global Positioning System sensor (GPS), a Bluetooth transceiver, a Wi-Fi transceiver, or an ultra-wide band location sensor.

43. The system of claim 39, wherein the defined dangerous geographical zone and the defined safe geographical zone are user-defined in both space and time.

44. The system of claim 43, wherein the defined dangerous geographical zone and the defined safe geographical zone change based on a time of day and/or a spatial proximity for both fixed and moving risks for addictive activities of the addict.

45. The system of claim 39, wherein the processor is configured to determine if the addict is in a place conducive to participating in addictive activities.

46. The system of claim 39, wherein the processor is configured to transmit geographical location information to one or more devices of members of an addiction support network of the addict to enable the members of the addiction support network to intervene and prevent the addict from participating in the addictive activities.

47. The system of claim 39, wherein the processor is configured to analyze the geographical location or proximity data, and determine when the addict has either entered the defined dangerous geographical zone or exited the defined safe geographical zone based on the geographical location or proximity data.

48. The system of claim 47, wherein the processor is configured to analyze the geographical location or proximity data in real-time.

49. A mobile system for identifying an addict that uses the mobile system to prevent an unauthorized use of the mobile system by another other than the addict, the mobile system comprising:
at least one biosensor configured to measure physiological data from the addict; and
a processor configured to send, via a communication system, a notification to one or more devices of the addict and/or of members of an addiction support network of the addict that the mobile system is not being worn by the addict based on the physiological data.

50. The mobile system of claim 49, wherein the processor is configured to compare the physiological data to baseline measurements, and determine if the mobile system is not being worn by the addict based on the comparison of the physiological data to the baseline measurements.

51. The mobile system of claim 49, wherein the processor is configured to analyze the physiological data, and determine when the mobile system is not being worn by the addict based on the physiological data.

52. The mobile system of claim 51, wherein the processor is configured to analyze the physiological data in real-time.

53. The mobile system of claim 49, wherein the processor is configured to communicate with the one or more devices via uni-directional or bi-directional communication to enable intervention and/or restoration of the mobile system on the addict.
